SEQUENCE LISTING (13 sequences)

SEQ ID NO: 1 - protein, 394 aa, Orthoflavivirus denguei
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGVSYVMCTGSFKLEKEVAETQHGTVLVQVKYEGTDAPCKIPFSSQDEKGVTQNGRLITANPIVTDKEKPVNIEAEPPFGESYLVVGAGEKALKLSWFKK

SEQ ID NO: 2 - protein, 394 aa, Orthoflavivirus denguei
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKK

SEQ ID NO: 3 - protein, 392 aa, Orthoflavivirus denguei
MRCVGVGNRDFVEGLSGATWVDVVLEHGGCVTTMAKNKPTLDIELQKTEATQLATLRKLCIEGKITNITTDSRCPTQGEATLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDVPCKIPFSTEDGQGKAHNGRLITANPVVTKKEEPVNIEAEPPFGESNIVIGIGDNALKINWYKK

SEQ ID NO: 4 - protein, 394 aa, Orthoflavivirus denguei
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFLCSGKITGNLVQIENLEYTVVVTVHNGDTPAVGNDTSHHGVTATITPRSPSVEVKLPDYGELTLDCEPRSGIDFIEMILMKMKKKTWLVHKQWFLDLPLPWTAGADTSEVHWNHKERMVTFKVPHAKRQDVTVLGSQEGAMHSALAGATEVDSGDGNHMFAGHLKCKVRMEKLRIKGMSYTMCSGKFSIDKEMAETQHGTAVVKVKYEGAGAPCKVPIEIRDVNKEKVVGRIISSTPFAENTNSVTNIELEPPFGDSYIVIGVGESALTLHWFRK

SEQ ID NO: 5 - protein, 394 aa, synthetic construct
MRCIGMSRRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKK

SEQ ID NO: 6 - protein, 394 aa, synthetic construct
MRCIGMSNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGGGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKK

SEQ ID NO: 7 - protein, 394 aa, synthetic construct
MRCIGMSRRDFVEGVSGGSWVDIVLEHGSCVTTMAKNKPTLDFELIKTEAKQPATLRKYCIEAKLTNTTTESRCPTQGEPSLNEEQDKRFVCKHSMVDRGGGNGCGLFGKGGIVTCAMFRCKKNMEGKVVQPENLEYTIVITPHSGEEHAVGNDTGKHGKEIKITPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVEPGQLKLNWFKK

SEQ ID NO: 8 - DNA, 1185 bp, synthetic construct
  source 1..1185: mol_type = other DNA
atgaggtgtattggaatgtctagacgggattttgtggagggagtctctggcgggagttgggtggatattgtcctggagcacggaagctgcgtgaccacaatggccaagaacaagcccaccctggacttcgagctgatcaagaccgaggccaagcagcctgccacactgcggaagtactgcatcgaggccaagctgaccaacaccacaaccgagagcagatgtcccacacagggcgagccttccctgaatgaggagcaggacaagcggttcgtgtgcaagcactctatggtggataggggatggggaaacggatgtggcctgttcggcaagggcggcatcgtgacctgcgccatgtttcggtgtaagaagaacatggagggcaaggtggtgcagcctgagaatctggagtacaccatcgtgatcacaccacacagcggagaggagcacgcagtgggaaatgacaccggcaagcacggcaaggagatcaagatcacaccacagagctccatcaccgaggccgagctgacaggctatggcacagtgaccatggagtgctccccaaggaccggcctggatttcaacgagatggtgctgctgcagatggagaataaggcctggctggtgcaccgccagtggtttctggacctgccactgccatggctgcctggagcagatacccagggctccaactggatccagaaggagacactggtgaccttcaagaatccacacgccaagaagcaggacgtggtggtgctgggctctcaggagggagcaatgcacacagccctgaccggagccacagagatccagatgtctagcggcaatctgctgttcaccggccacctgaagtgccggctgagaatggataagctgcagctgaagggcatgagctactccatgtgcaccggcaagtttaaggtggtgaaggagatcgccgagacacagcacggcacaatcgtgatcagggtgcagtatgagggcgacggcagcccttgtaagatcccattcgagatcatggatctggagaagcggcacgtgctgggcagactgatcaccgtgaacccaatcgtgacagagaaggactctcccgtgaatatcgaggccgagcccccttttggcgatagctacatcatcatcggcgtggagcccggccagctgaagctgaactggttcaagaagtaa

SEQ ID NO: 9 - DNA, 1185 bp, synthetic construct
  source 1..1185: mol_type = other DNA
atgaggtgtattggaatgtctaaccgggattttgtggagggagtctctggcgggagttgggtggatattgtcctggagcacggaagctgcgtgaccacaatggccaagaacaagcccaccctggacttcgagctgatcaagaccgaggccaagcagcctgccacactgcggaagtactgcatcgaggccaagctgaccaacaccacaaccgagagcagatgtcccacacagggcgagccttccctgaatgaggagcaggacaagcggttcgtgtgcaagcactctatggtggataggggaggaggaaacggatgtggcctgttcggcaagggcggcatcgtgacctgcgccatgtttcggtgtaagaagaacatggagggcaaggtggtgcagcctgagaatctggagtacaccatcgtgatcacaccacacagcggagaggagcacgcagtgggaaatgacaccggcaagcacggcaaggagatcaagatcacaccacagagctccatcaccgaggccgagctgacaggctatggcacagtgaccatggagtgctccccaaggaccggcctggatttcaacgagatggtgctgctgcagatggagaataaggcctggctggtgcaccgccagtggtttctggacctgccactgccatggctgcctggagcagatacccagggctccaactggatccagaaggagacactggtgaccttcaagaatccacacgccaagaagcaggacgtggtggtgctgggctctcaggagggagcaatgcacacagccctgaccggagccacagagatccagatgtctagcggcaatctgctgttcaccggccacctgaagtgccggctgagaatggataagctgcagctgaagggcatgagctactccatgtgcaccggcaagtttaaggtggtgaaggagatcgccgagacacagcacggcacaatcgtgatcagggtgcagtatgagggcgacggcagcccttgtaagatcccattcgagatcatggatctggagaagcggcacgtgctgggcagactgatcaccgtgaacccaatcgtgacagagaaggactctcccgtgaatatcgaggccgagcccccttttggcgatagctacatcatcatcggcgtggagcccggccagctgaagctgaactggttcaagaagtaa

SEQ ID NO: 10 - DNA, 1185 bp, synthetic construct
  source 1..1185: mol_type = other DNA
atgaggtgtattggaatgtctagacgggattttgtggagggagtctctggcgggagttgggtggatattgtcctggagcacggaagctgcgtgaccacaatggccaagaacaagcccaccctggacttcgagctgatcaagaccgaggccaagcagcctgccacactgcggaagtactgcatcgaggccaagctgaccaacaccacaaccgagagcagatgtcccacacagggcgagccttccctgaatgaggagcaggacaagcggttcgtgtgcaagcactctatggtggataggggaggaggaaacggatgtggcctgttcggcaagggcggcatcgtgacctgcgccatgtttcggtgtaagaagaacatggagggcaaggtggtgcagcctgagaatctggagtacaccatcgtgatcacaccacacagcggagaggagcacgcagtgggaaatgacaccggcaagcacggcaaggagatcaagatcacaccacagagctccatcaccgaggccgagctgacaggctatggcacagtgaccatggagtgctccccaaggaccggcctggatttcaacgagatggtgctgctgcagatggagaataaggcctggctggtgcaccgccagtggtttctggacctgccactgccatggctgcctggagcagatacccagggctccaactggatccagaaggagacactggtgaccttcaagaatccacacgccaagaagcaggacgtggtggtgctgggctctcaggagggagcaatgcacacagccctgaccggagccacagagatccagatgtctagcggcaatctgctgttcaccggccacctgaagtgccggctgagaatggataagctgcagctgaagggcatgagctactccatgtgcaccggcaagtttaaggtggtgaaggagatcgccgagacacagcacggcacaatcgtgatcagggtgcagtatgagggcgacggcagcccttgtaagatcccattcgagatcatggatctggagaagcggcacgtgctgggcagactgatcaccgtgaacccaatcgtgacagagaaggactctcccgtgaatatcgaggccgagcccccttttggcgatagctacatcatcatcggcgtggagcccggccagctgaagctgaactggttcaagaagtaa

SEQ ID NO: 11 - DNA, 1520 bp, synthetic construct
  source 1..1520: mol_type = other DNA
gggaaataagagagaaaagaagagtaagaagaaatataagagccaccatggagacagacacactcctgctatgggtactgctgctctgggttccaggttccaccggtgacatgaggtgtattggaatgtctagacgggattttgtggagggagtctctggcgggagttgggtggatattgtcctggagcacggaagctgcgtgaccacaatggccaagaacaagcccaccctggacttcgagctgatcaagaccgaggccaagcagcctgccacactgcggaagtactgcatcgaggccaagctgaccaacaccacaaccgagagcagatgtcccacacagggcgagccttccctgaatgaggagcaggacaagcggttcgtgtgcaagcactctatggtggataggggatggggaaacggatgtggcctgttcggcaagggcggcatcgtgacctgcgccatgtttcggtgtaagaagaacatggagggcaaggtggtgcagcctgagaatctggagtacaccatcgtgatcacaccacacagcggagaggagcacgcagtgggaaatgacaccggcaagcacggcaaggagatcaagatcacaccacagagctccatcaccgaggccgagctgacaggctatggcacagtgaccatggagtgctccccaaggaccggcctggatttcaacgagatggtgctgctgcagatggagaataaggcctggctggtgcaccgccagtggtttctggacctgccactgccatggctgcctggagcagatacccagggctccaactggatccagaaggagacactggtgaccttcaagaatccacacgccaagaagcaggacgtggtggtgctgggctctcaggagggagcaatgcacacagccctgaccggagccacagagatccagatgtctagcggcaatctgctgttcaccggccacctgaagtgccggctgagaatggataagctgcagctgaagggcatgagctactccatgtgcaccggcaagtttaaggtggtgaaggagatcgccgagacacagcacggcacaatcgtgatcagggtgcagtatgagggcgacggcagcccttgtaagatcccattcgagatcatggatctggagaagcggcacgtgctgggcagactgatcaccgtgaacccaatcgtgacagagaaggactctcccgtgaatatcgaggccgagcccccttttggcgatagctacatcatcatcggcgtggagcccggccagctgaagctgaactggttcaagaagtaaggtacctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 1520 bp, synthetic construct
  source 1..1520: mol_type = other DNA
gggaaataagagagaaaagaagagtaagaagaaatataagagccaccatggagacagacacactcctgctatgggtactgctgctctgggttccaggttccaccggtgacatgaggtgtattggaatgtctaaccgggattttgtggagggagtctctggcgggagttgggtggatattgtcctggagcacggaagctgcgtgaccacaatggccaagaacaagcccaccctggacttcgagctgatcaagaccgaggccaagcagcctgccacactgcggaagtactgcatcgaggccaagctgaccaacaccacaaccgagagcagatgtcccacacagggcgagccttccctgaatgaggagcaggacaagcggttcgtgtgcaagcactctatggtggataggggaggaggaaacggatgtggcctgttcggcaagggcggcatcgtgacctgcgccatgtttcggtgtaagaagaacatggagggcaaggtggtgcagcctgagaatctggagtacaccatcgtgatcacaccacacagcggagaggagcacgcagtgggaaatgacaccggcaagcacggcaaggagatcaagatcacaccacagagctccatcaccgaggccgagctgacaggctatggcacagtgaccatggagtgctccccaaggaccggcctggatttcaacgagatggtgctgctgcagatggagaataaggcctggctggtgcaccgccagtggtttctggacctgccactgccatggctgcctggagcagatacccagggctccaactggatccagaaggagacactggtgaccttcaagaatccacacgccaagaagcaggacgtggtggtgctgggctctcaggagggagcaatgcacacagccctgaccggagccacagagatccagatgtctagcggcaatctgctgttcaccggccacctgaagtgccggctgagaatggataagctgcagctgaagggcatgagctactccatgtgcaccggcaagtttaaggtggtgaaggagatcgccgagacacagcacggcacaatcgtgatcagggtgcagtatgagggcgacggcagcccttgtaagatcccattcgagatcatggatctggagaagcggcacgtgctgggcagactgatcaccgtgaacccaatcgtgacagagaaggactctcccgtgaatatcgaggccgagcccccttttggcgatagctacatcatcatcggcgtggagcccggccagctgaagctgaactggttcaagaagtaaggtacctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 1520 bp, synthetic construct
  source 1..1520: mol_type = other DNA
gggaaataagagagaaaagaagagtaagaagaaatataagagccaccatggagacagacacactcctgctatgggtactgctgctctgggttccaggttccaccggtgacatgaggtgtattggaatgtctagacgggattttgtggagggagtctctggcgggagttgggtggatattgtcctggagcacggaagctgcgtgaccacaatggccaagaacaagcccaccctggacttcgagctgatcaagaccgaggccaagcagcctgccacactgcggaagtactgcatcgaggccaagctgaccaacaccacaaccgagagcagatgtcccacacagggcgagccttccctgaatgaggagcaggacaagcggttcgtgtgcaagcactctatggtggataggggaggaggaaacggatgtggcctgttcggcaagggcggcatcgtgacctgcgccatgtttcggtgtaagaagaacatggagggcaaggtggtgcagcctgagaatctggagtacaccatcgtgatcacaccacacagcggagaggagcacgcagtgggaaatgacaccggcaagcacggcaaggagatcaagatcacaccacagagctccatcaccgaggccgagctgacaggctatggcacagtgaccatggagtgctccccaaggaccggcctggatttcaacgagatggtgctgctgcagatggagaataaggcctggctggtgcaccgccagtggtttctggacctgccactgccatggctgcctggagcagatacccagggctccaactggatccagaaggagacactggtgaccttcaagaatccacacgccaagaagcaggacgtggtggtgctgggctctcaggagggagcaatgcacacagccctgaccggagccacagagatccagatgtctagcggcaatctgctgttcaccggccacctgaagtgccggctgagaatggataagctgcagctgaagggcatgagctactccatgtgcaccggcaagtttaaggtggtgaaggagatcgccgagacacagcacggcacaatcgtgatcagggtgcagtatgagggcgacggcagcccttgtaagatcccattcgagatcatggatctggagaagcggcacgtgctgggcagactgatcaccgtgaacccaatcgtgacagagaaggactctcccgtgaatatcgaggccgagcccccttttggcgatagctacatcatcatcggcgtggagcccggccagctgaagctgaactggttcaagaagtaaggtacctgataataggctggagcctcggtggccatgcttcttgccccttgggcctccccccagcccctcctccccttcctgcacccgtacccccgtggtctttgaataaagtctgagtgggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0232788 A1
(43) Pub. Date: Aug. 13, 2026

(54) DENGUE VIRUS MRNA VACCINE AND METHOD AGAINST DENGUE VIRUS INFECTION USING THE SAME

(71) Applicant: ACADEMIA SINICA, Taipei City (TW)

(72) Inventors: Han-Chung WU, Taipei City (TW); Shih-Chieh SU, New Taipei City (TW); Hsiu-Ting LIN, New Taipei City (TW)

(73) Assignee: ACADEMIA SINICA, Taipei City (TW)

(21) Appl. No.: 19/156,524

(22) PCT Filed: Feb. 15, 2024
(86) PCT No.: PCT/US2024/015894

§ 371 Date: Aug. 14, 2025

Related U.S. Application Data
(60) Provisional application No. 63/485,090, filed on Feb. 15, 2023.

(51) Int. Cl. A61K 39/12; A61K 9/1272; A61K 9/51; A61K 39/00; A61P 31/14; B82Y 5/00; C07K 14/005; C12N 7/00; C12N 15/88

(52) U.S. Cl. CPC A61K 39/12; A61K 9/1272; A61K 9/5123; A61P 31/14; C07K 14/005; C12N 7/00; C12N 15/88; A61K 2039/53; A61K 2039/545; A61K 2039/55555; A61K 2039/575; B82Y 5/00; C12N 2770/24122; C12N 2770/24134

(57) ABSTRACT

The present invention relates to a dengue virus messenger ribonucleic acid (mRNA) vaccine. In particular, the vaccine includes an mRNA encoding a mutant envelop (E) protein of dengue virus at position 8 and/or 101 formulated in lipid nanoparticles. The mRNA vaccine of the present invention is safe and effective, which causes enhanced production of neutralizing antibodies against multiple serotypes of dengue virus and reduced antibody dependent enhancement (ADE) response.

### RELATED APPLICATIONS

This application claims the benefit of U.S. provisional application No. 63/485,090, filed Feb. 15, 2023 under 35 U.S.C. § 119, the entire content of which is incorporated herein by reference.

### TECHNOLOGY FIELD

The present invention relates to a dengue virus messenger ribonucleic acid (mRNA) vaccine. In particular, the vaccine includes an mRNA encoding a mutant envelop (E) protein of dengue virus at position 8 and/or 101 formulated in lipid nanoparticles. The mRNA vaccine of the present invention is safe and effective, which causes enhanced production of neutralizing antibodies against multiple serotypes of dengue virus and reduced antibody dependent enhancement (ADE) response.

### BACKGROUND OF THE INVENTION

Dengue is an arboviral (arthropod-borne virus) disease caused by the dengue virus (DENV), which is mainly transmitted by Aedes aegypti mosquitoes. DENV strains belong to the Flaviviridae family and are categorized into four genetically distinct serotypes (DENV1 to DENV4). These serotypes have spread globally and constitute a growing threat to public health (Wilder-Smith, 2020). Infection with any of the four DENV serotypes causes disease that may present as asymptomatic to severe dengue symptoms, or even potentially fatal shock syndrome. Importantly, primary infection with DENV can provide protective immunity without causing serious illness, but subsequent infections with any serotype carry increased risk of severe dengue symptoms or shock syndrome (Wollner et al., 2021). The incidence of DENV infection has increased by eight-fold in the last two decades (WHO, 2022), with serious illness often seen in young children (Guzman et al., 2010). Thus, intensive efforts have been made in the last 30 years to develop a vaccine against the virus. One major obstacle to producing a vaccine for this virus is that the different serotypes are cross-reactive, and antibody-dependent enhancement (ADE) can potentially reduce vaccine efficiency and increase the risk to health (Bhatt et al., 2021). The DENV genome consists of a positive RNA strand of ~11 kb, which is translated into a single polyprotein that comprises ten proteins; three are structural proteins [capsid (C), premembrane (prM) and envelope (E)], and seven are non-structural proteins (NS1 NS2A, NS2B, NS3, NS4A, NS4B, and NS5) (Guzman et al., 2016). Many attempts have been made to utilize different structural or non-structural proteins of DENV in order to generate dengue vaccines (Zhang et al., 2020).

To date, there is only one vaccine, Dengyaxia, that is licensed in endemic countries. This vaccine was developed by using a live attenuated vaccine for yellow fever as a backbone and replacing the yellow fever genomic sequence with DENV E and prM of each serotype. Although Dengyaxia is licensed in endemic countries, it showed low efficacy in phase III trials in dengue naïve individuals. Furthermore, the vaccine stimulates low levels of neutralizing antibodies against DENV, so a booster dose is required (Amorim and Birbrair, 2022; Henein et al., 2021). Reasons for its low efficiency may be related to the absence of non-structural DENV proteins and/or poor balance of virus replication among the four serotypes (Wilder-Smith, 2020). These characteristics may limit vaccine efficacy by lowering production of neutralizing antibodies and increasing the risk of antibody dependent enhancement (ADE) response (Thomas and Yoon, 2019).

Indonesian authorities have recently approved Qdenga as a vaccine for people between the ages of 6 and 45. An engineered tetravalent dengue vaccine is a live attenuated virus using DENV-2 as its backbone and incorporating genes from other three serotypes into it. It is, however, important to note that the complete data on DENV1, 3, and 4 serotypes do not exist, which reflects the danger of ADE response against these serotypes, and may lead to some serious ADE response in the near future (Mallapaty, 2022). Nevertheless, the limitations of this licensed vaccine have given researchers a clear idea about the important issues in designing future products. For example, the foremost goal of any new DENV vaccine design should be to achieve a low potential ADE response along with high production of neutralizing antibodies.

### SUMMARY OF THE INVENTION

The present invention, is based, at least in part, on the development of a messenger ribonucleic acid (mRNA) encoding a mutant envelop (E) protein of dengue virus at position 8 and/or 101, formulated in lipid nanoparticles (LNP), for vaccinating a subject against dengue virus infection. Surprisingly, immunization of animals with the mRNA-LNP causes enhanced production of neutralizing antibodies against multiple serotypes of dengue virus and reduced antibody dependent enhancement (ADE) response.

Accordingly, in one aspect, the present invention provides a composition, which comprises a messenger ribonucleic acid (mRNA) comprising an open reading frame encoding a dengue virus B protein variant formulated in a lipid nanoparticle, wherein the E protein variant comprises amino acid mutation at position 8 and/or position 101 of SEQ ID NO: 2.

In some embodiments, the one or more amino acid mutation is selected from the group consisting of
(a) an amino acid substitution of the 8^{th} Asn (N) residue;
(b) an amino acid substitution of the 101^{th} Try (W) residue; and
(c) an amino acid substitution of the 8^{th} Asn (N) residue and an amino acid substitution of the 101^{th} Try (W) residue.

In some embodiments, the one or more amino acid mutation is selected from the group consisting of
(a) an amino acid substitution of the 8^{th} Asn (N) residue with Arg (R);
(b) an amino acid substitution of the 101^{th} (W) residue with Gly (G); and
(c) an amino acid substitution of the 8^{th} Asn (N) residue with Arg (R) and an amino acid substitution of the 101^{th} Try (W) residue with Gly (G).

In some embodiments, the mRNA encodes an amino acid sequence set forth in SEQ ID NO: 5, 6 or 7.

In some embodiments, the mRNA comprises an nucleic acid sequence set forth in SEQ ID NO: 8, 9 or 10.

In some embodiments, the mRNA further comprises a 5′ untranslated region (UTR) and a 3′UTR.

In some embodiments, the mRNA comprises SEQ ID NO: 11, 12 or 13.

In some embodiments, the mRNA further comprises
a 5′ cap analog; and/or
a poly(A) tail.

In some embodiments, the 5′ cap analog is 7 mG(5′)ppp(5′)NlmpNp.

In some embodiments, the mRNA comprises a chemical modification.

In some embodiments, the chemical modification is a 1-methylpseudouridine modification.

In some embodiments, the lipid nanoparticle comprises a cationic lipid, a non-cationic lipid, a sterol and a polyethylene glycol (PEG)-modified lipid.

In some embodiments, the lipid nanoparticle comprises 20-70% cationic lipid, 5-45% non-cationic lipid, 20-55% sterol, and 0.5-15% PEG-modified lipid.

In some embodiments, the lipid nanoparticle comprises 50% cationic lipid, 10% non-cationic lipid, 38.5% sterol and 1.5% PEG-modified lipid.

In some embodiments, the lipid nanoparticle comprises a lipid bilayer surrounding a core where the mRNA is encapsulated.

In another aspect, the present invention provides a vaccine against dengue virus infection which comprises an effective amount of a composition comprising an open reading frame encoding a dengue virus E protein variant formulated in a lipid nanoparticle as described herein and a physiologically acceptable vehicle.

In a further aspect, the present invention provides a composition comprising an open reading frame encoding a dengue virus E protein variant formulated in a lipid nanoparticle as described herein for use in immunization against dengue virus infection in a subject in need thereof. The present invention also provides use of a composition comprising an open reading frame encoding a dengue virus E protein variant formulated in a lipid nanoparticle as described herein for manufacturing a medicament for immunization against dengue virus infection in a subject in need thereof. The present invention further provides a method for immunization against dengue virus infection in a subject in need thereof, comprising administering to the subject in need thereof a therapeutically effective amount of a composition comprising an open reading frame encoding a dengue virus E protein variant formulated in a lipid nanoparticle as described herein.

In some embodiments, the dengue virus infection is caused by dengue virus serotype 1 (DENV1), dengue virus serotype 2 (DENV2), dengue virus serotype 3 (DENV3) and/or dengue virus serotype 4 (DENV4).

In some embodiments, the amount of the composition is effective in inducing neutralizing antibodies against the dengue virus and/or alleviating antibody dependent enhancement of the dengue virus infection.

In some embodiments, the composition is administered to the subject once or more than once.

The details of one or more embodiments of the invention are set forth in the description below. Other features or advantages of the present invention will be apparent from the following detailed description of several embodiments, and also from the appending claims

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A to ID show synthesis and physicochemical characterization of mRNA-LNP. The illustrations depict mRNA synthesis by IVT (FIG. 1A) and mRNA-LNP synthesis using a microfluidic device (FIG. 1B). Agarose gel retardation assay: Lanes 1~4 represent the free mRNA, lanes S-8 represent the mRNA-LNP complex, and lanes 9-12 represent the mRNA-LNP treated with 1% Triton X-100 prior to loading (FIG. 1C). Cryo-EM image shows mRNA-LNP complex (FIG. 1D).

FIGS. 2A to 2C show functional analysis of mRNA-LNP complex. The schematic image depicts the transfection of 293T cells with mRNA-LNP (FIG. 2A). Quantification of protein expression in transfected cells was performed using flow cytometry (FIG. 2B). The bar graph represents the protein expression level (FIG. 2C).

FIGS. 3A to 3E show expression and immunogenicity in BALB/c mice. BALB/c mice were immunized with 10 μg of the mRNA-LNP complex by intramuscular injection (FIG. 3A). Serum was collected after 6 weeks of prime-boost doses, and dengue virus-specific antibody responses were analyzed by ELISA. The ELISA plates were coated with specific recombinant proteins; DENV1 (FIG. 3B), DENV2 (FIG. 3C), DENV3 (FIG. 3D), and DENV4 (FIG. 3E).

FIGS. 4A to 4D show neutralizing antibody titers and antibody-dependent enhancement response against DENV2 in immune sera. The PRNT assay was performed using mouse sera collected six weeks after the initial immunization (FIG. 4A). BHK-21 cells were co-incubated with the DENV2 virus and different dilutions of immunized mice sera. Plaques were fixed and stained at 5 days post-infection. Representative images of PRNT assay (FIG. 4B). The graph shows % of plaques by dilution, and the dashed line represents the PRNT₅₀ value (FIG. 4C). Antibody-dependent enhancement (ADE) of DENV2 replication was analyzed by the PRNT assay performed on the supernatant of K562 cells that had been treated with serially diluted sera from immunized mice (FIG. 4D).

FIGS. 5A to 5D show the antibody expression and immunogenicity in BALB/c mice immunized with E₁₋₃₉₄-N8R and E₁₋₃₉₄-WT mRNA-LNP containing AS-CL-28 ionizable lipid. BALB/c mice were immunized with 10 μg of the mRNA-LNP complex by intramuscular injection (FIG. 5A). Serum was collected after 6 weeks of the first immunization, and dengue virus-specific antibody responses were analyzed by ELISA. ELISA plates were coated with DENV2 recombinant proteins, and sera from immunized mice were evaluated (FIG. 5B). The graph shows % of plaques, and the dashed line represents the 50% of viral inhibition (FIG. 5C). ADE of DENV2 replication was analyzed by PRNT assay (FIG. 5D). In vitro assessment of the ADE response to DENV2 replication.

FIG. 6 Shows the neutralizing activity and protective effect of immunized sera on suckling mice. Serially diluted immunized sera were incubated with 1×10⁴ pfu (25-fold LD50) of DENV2 16681 for 0.5 hours at 4° C. Two-day-old suckling mice were inoculated with 20 μl of the mixture via intracranial (i.e.) injection. After the challenge, survival was monitored for 28 days.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of #10%, particularly ±5%, around the cited value.

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “complementary” refers to the topological compatibility or matching together of interacting surfaces of two polynucleotides. A first polynucleotide is complementary to a second polynucleotide when the nucleotide sequence of the first polynucleotide is identical to the nucleotide sequence of the polynucleotide binding partner of the second polynucleotide. Thus, the polynucleotide whose sequence 5′-GATAT-3′ is complementary to a polynucleotide whose sequence is 5′-ATATC-3′″.

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. As used herein, a “coding sequence” or a sequence “encoding” an expression product, such as an RNA or polypeptide, is a nucleotide sequence that, when expressed, results in the production of that RNA or polypeptide i.e., the nucleotide sequence encodes an amino acid sequence for that polypeptide. A coding sequence may include a start codon (usually ATG) and a stop codon (e.g. TAA, TAG or TGA). It may constitute an “uninterrupted coding sequence” (i.e., lacking an intron, such as in a cDNA) or it may include one or more introns bounded by splice junctions. An “open reading frame (ORF)” is the coding sequence uninterrupted by introns between the start codon and the stop codon that encodes an amino acid sequence. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

As used herein, the term “substantially identical” refers to two sequences having 80% or more, preferably 85% or more, more preferably 90% or more, even more preferably 95% or more homology. To determine the percent identity of two sequences, the sequences can be aligned for optimal comparison purpose. In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

Dengue virus is a single-stranded RNA virus that is a member of the family Flaviviridae, genus Flavivirus. Dengue virus includes different serotypes, including dengue virus serotype 1 (DENV1), dengue virus serotype 2 (DENV2), dengue virus serotype 3 (DENV3) and dengue virus serotype 4 (DENV4). Those distinct dengue virus serotypes are genetically related but antigenically distinct. The serotypes can be determined by conventional methods known in the art such as RT-PCR using specific primer sets to amplify serotype-specific fragments from the regions encoding the capsid and membrane proteins of dengue virus.

As used herein, a dengue virus envelop (E) protein variant includes amino acid mutation at position 8 and/or position 101, corresponding to the amin acid residues 1-394 of wild-type DENV2. In particular, the amino acid mutation includes an amino acid substitution. In some embodiments, the amino acid mutation includes a substitution of the 81 Asn residue (N8 substitution). In some embodiments, the amino acid mutation includes a substitution of the 101^{th} Try residue (W101 substitution). In some embodiments, the amino acid mutation includes a substitution of the 8^{th} Asn residue and an amino acid substitution of the 101^{th} Try residue (N8+W101 substitution). In certain embodiments, the 8^{th} Asn residue is substituted with Arg (N8R). In certain embodiments, the 101^{th} Try residue is substituted with Gly (W101G).

As used herein, the term “chemical modification” means modification to adenosine (A), guanosine (G), thymidine (T), uridine (U), or cytidine (C) ribonucleosides or deoxyribnucleosides, in their position, pattern, percent and/or population. Polynucleotides may comprise modifications that are naturally-occurring, non-naturally-occurring or the polynucleotide may comprise a combination of naturally-occurring and non-naturally-occurring modifications. The modifications may be present on an internucleotide linkages, purine or pyrimidine bases, or sugars. In some instances, non-natural modified nucleotides are introduced during synthesis or post-synthesis of the polynucleotides to achieve desired functions or properties. The modification may be introduced via chemical synthesis or via a polymerase enzyme at the terminal of a chain or anywhere else in the chain. Any of the regions of a polynucleotide may be chemically modified. Polynucleotides may be partially or fully modified along the entire length of the molecule.

In some embodiments, the chemical modification is at nucleobases in the polyribonucleotides (e.g., RNA, such as mRNA). In some instances, modified nucleobases in the polyribonucleotides include, but not limited to, α-thio-adenosine, α-thio-guanosine, pseudouridine (ψ), 1-methyl-pseudouridine (m1ψ), 1-ethyl-pseudouridine (e1ψ), 5-methoxy-uridine (mo5U), and 5-methyl-cytidine (m5C).

As used herein, 5′-cap is typically a modified nucleotide entity adding to 5′-end of an RNA which can inhibit degradation of the mRNA. A 5′-cap may typically be formed by a modified nucleotide (cap analog), particularly by a derivative of a guanine nucleotide. Preferably, the 5′-cap is linked to the 5′-terminus via a 5′-5′-triphosphate linkage. A 5′-cap may be methylated, e.g. m7GpppN (e.g. m7G(5′)ppp(5′)G(m7G)), wherein N is the terminal 5′ nucleotide of the nucleic acid carrying the 5′-cap, typically the 5′-end of an RNA.

As used herein, a 5′ untranslated region″ (5′UTR) refers to a region of an mRNA that is upstream of the coding sequence on the 5′end of the mature mRNA that does not encode a polypeptide.

As used herein, a 3′ untranslated region″ (3′UTR) refers to a region of an mRNA that is downstream of the coding sequence on the 5′end of the mature mRNA that does not encode a polypeptide

A “polyA tail” is a region of mRNA that is downstream from the 3′ UTR and contains numerous, consecutive adenosine monophosphates. A polyA tail may contain 10 to 300 adenosine monophosphates. It can protect mRNA from degradation.

As used herein, the term “lipid-nanoparticle (LNP) delivery” indicates the transmembrane delivery of macromolecules, such as nucleic acids, proteins, etc., into cells through lipid nanoparticles. Typically, a lipid nanoparticle comprises a cationic lipid, a non-cationic lipid, a sterol and a polyethylene glycol (PEG)-modified lipid. In particular, lipid nanoparticles may be formed by mixing two phases, including an ethanol phase containing a cationic lipid, a non-cationic lipid, sterol, and PEG-modified lipid, and an acidic aqueous phase containing macromolecules such as nucleic acids and proteins. Typically, a LNP forms a lipid bilayer surrounding a core where RNA is encapsulated, which can enter the cytoplasm via endocytosis.

Cationic lipids useful in the present invention can be a lipid which carries a net positive charge at a selected pH, such as physiological pH. Examples of suitable cationic lipids include, but are not limited to, dilinoleyl-methyl-4-dimethylaminobutyrate (DLin-MC3-DMA), 2,2-dilinoleyl-4-dimethylaminoethyl-[1,3]-dioxolane (DLin-KC2-DMA), di((Z)-non-2-en-1-yl) 9-((4-(dimethylamino)butanoyl)oxy) heptadecanedioate (L319), N,N-dimethyl-1-[(1S,2R)-2-octylcyclopropyl]heptadecan-8-amine (L530) and (12Z,15Z)—N,N-dimethyl-2-nonylhenicosa-12,15-dien-1-amine (L608).

Non-cationic lipids useful in the present invention can be a variety of neutral uncharged, zwitterionic or anionic lipids capable of producing a stable complex. Examples of noncationic lipids useful in the present invention include phospholipid-related materials, such as 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), (1,2-dimyristoyl-sn-glycero-3-phosphoethanolamine (DMPE), 2-dioleoyl-sn-glycero-3-phospho-(1′-rac-glycerol) (DOPG), 1,2-dioleoyl-sw-glycero-3-phosphoethanolamine (DOPE), 1,2-distearoyl-s«-glycero-3-phosphoethanolamine (DSPE), 1,2-dilauroyl-sw-glycero-3-phosphoethanolamine (DLPE) and 1,2-dipalmitoyl-sw-glycero-3-phospho-L-serine (DPPS).

A lipid-nanoparticle may include a sterol as the structural lipids. Any suitable sterol may be used in the present invention, such as those selected from the group consisting of cholesterol, ergosterol, campesterol, oxysterol, antrosterol, desmosterol, nicasterol, sitosterol, stigmasterol and mixtures thereof. A typical example is cholesterol.

A lipid-nanoparticle may include one or more PEG-modified lipids. A PEG lipid is a lipid modified with polyethylene glycol. In some embodiments, the PEG-lipid includes, but not limited to 1,2-dimyristoyl-sn-glycerol methoxypoly ethylene glycol (PEG-DMG), PEG-disteryl glycerol (PEG-DSG), 1,2-distearoyl-sn-glycero-3-phosphoethanolamine-N-[amino (poly ethylene glycol)] (PEG-DSPE), PEG-diacylglycamide (PEG-DAG) and PEG-dipalmitoyl phosphatidylethanolamine (PEG-DPPE).

In some embodiments, a lipid-nanoparticle as described herein comprises a lipid mixture in molar ratios of 20-70% cationic lipid: 5-45% non-cationic (neutral) lipid: 20-55% cholesterol: 0.5-15% PEG-modified lipid. In some embodiments, a lipid-nanoparticle as described herein comprises a lipid mixture in molar ratios of 20-60% cationic lipid: 5-25% non-cationic (neutral) lipid: 25-55% cholesterol: 0.5-15% PEG-modified lipid. In one particular example, the molar lipid ratio is 50:10:38.5:1.5 (mol %, cationic lipid:non-cationic lipid:cholesterol:PEG-modified lipid.

In some embodiments, a lipid-nanoparticle as described herein may has a diameter from about 10 to about 500 nm, particularly around 100 nm.

As used herein, the terms “subject,” “individual” and “patient,” used interchangeably herein, refer to a mammalian subject for whom diagnosis, prognosis, treatment, or therapy is needed, particularly humans. Other subjects may include cattle, dogs, cats, guinea pigs, rabbits, rats, mice, horses, and so on.

As used herein, the term “treatment” refers to the application or administration of one or more active agents to a subject afflicted with a disorder, a symptom or condition of the disorder, or a progression of the disorder, with the purpose to cure, heal, alleviate, relieve, alter, remedy, ameliorate, improve, or affect the disorder, the symptom or condition of the disorder, the disabilities induced by the disorder, or the progression or predisposition of the disorder.

As used herein, the term “effective amount” refers to the amount of an active ingredient to confer a desired biological effect in a treated subject or cell. The effective amount may change depending on various reasons, such as administration route and frequency, body weight and species of the individual receiving said pharmaceutical, and purpose of administration. Persons skilled in the art may determine the dosage in each case based on the disclosure herein, established methods, and their own experience.

In the present invention, a LNP including a mRNA encoding a mutant E protein of dengue virus as described herein can be formulated with a physiologically acceptable carrier for the production of vaccines. As used herein, “physiologically acceptable” means that the carrier is compatible with an active ingredient in the composition, and preferably can stabilize said active ingredient and is safe to the receiving individual. Such physiologically acceptable carriers are well known in the art. In some embodiments, a LNP as described herein is formulated and administered as a sterile solution while it is also possible to utilize lyophilized preparations. Sterile solutions can be lyophilized or filled into pharmaceutical dosage containers. The pH of the solution generally is in the range of pH 3.0 to 9.5, e.g pH 5.0 to 7.5. The LNP typically is in a solution having a suitable pharmaceutically acceptable buffer. In certain embodiments, the LNP may be formulated into an injectable preparation.

According to the present invention, a composition comprising a LNP including a mRNA encoding a mutant E protein of dengue virus as described herein is useful in vaccinating a subject in need thereof. In particular, the vaccination induces protective immunity against dengue virus infection. In some embodiments, the dengue virus infection is caused by one or more serotypes, selected from the group consisting of dengue virus serotype 1 (DENV1), dengue virus serotype 2 (DENV2), dengue virus serotype 3 (DENV3), dengue virus serotype 4 (DENV4), or any combination thereof. In some embodiments, the protective immunity provides efficacious levels of neutralizing antibodies against the dengue virus infection in the blood or serum of a vaccinated subject. In particular embodiments, the protective immunity causes reduced levels of antibody dependent enhancement of the dengue virus infection in a vaccinated subject.

In the present invention, administration of a composition or a vaccine can be performed using standard routes of administration. Exemplified administration includes intramuscular administration, intradermal administration and subcutaneous administration. The administration may be performed as a single dose, or as a prime and boosting. In some embodiments, the composition of the present invention is administered to the subject once or more than once, such as twice, three times, four times, five times, six times or more. The period of time between prime and boost is generally one (1) week, two (2) weeks, four (4) weeks, six (6) weeks or eight (8) weeks, preferably 4 weeks or 8 weeks.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### EXAMPLES

Here, we mutated the sequence of amino acids in the wild-type (WT) E structural proteins at positions 8 and 101. The arginine at position 8 was replaced with asparagine (N8R), while the glycine at position 101 was replaced with tryptophan (G101W). These mutations were made in order to enhance the production of neutralizing antibodies and reduce ADE response. The mRNAs were produced via in vitro transcription (IVT). Then, the modified mRNAs and WT DENV E mRNA were encapsulated in lipid nanoparticles (LNPs). We evaluated the sera of animals immunized with the modified mRNA sequences that possessed antibodies. The binding efficacy of sera was evaluated against all four DENV serotypes by using ELISA assay. Meanwhile, the levels of neutralizing antibody titers were examined by the plaque reduction neutralization titer (PRNT) assay. Finally, the ADE response of immunized mice sera toward DENV2 was monitored by testing viral replication activity in Fcγ receptor-positive K562 cells following the PRNT assay. Our data showed that sera of animals immunized with the modified mRNA sequence carried high levels of antibodies that efficiently bind to DENV1-3 serotypes and had low ADE response against DENV2. As a result of these findings, mRNA-LNP immunization against mutant E protein appears to be a promising approach for creating effective dengue vaccines with low potential for ADE.

### 1. Material and Methods

1.1 Production of Modified IVT mRNA

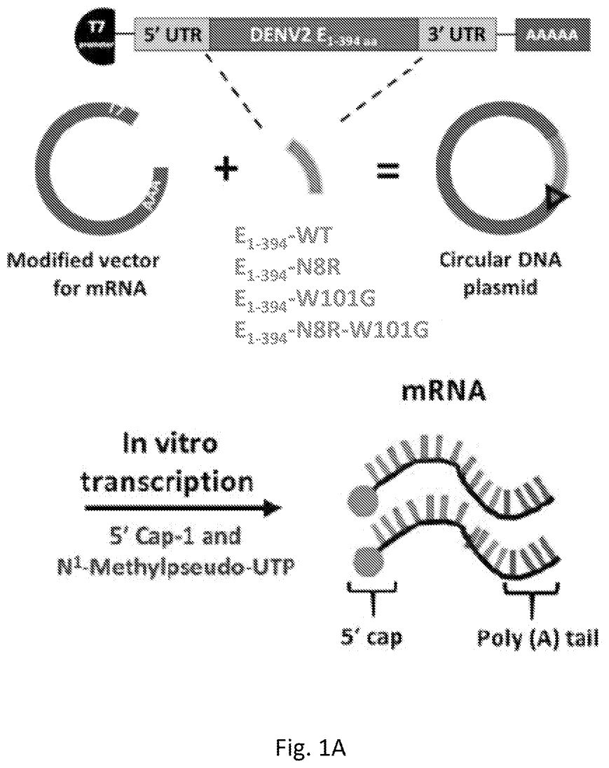

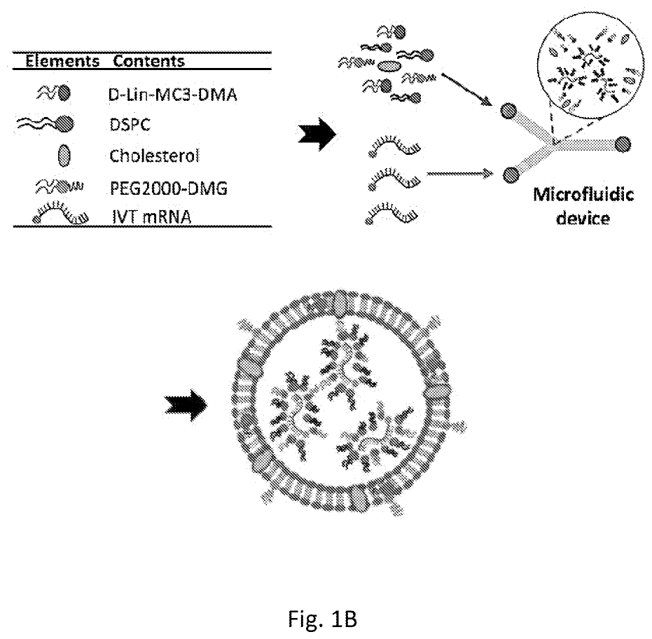

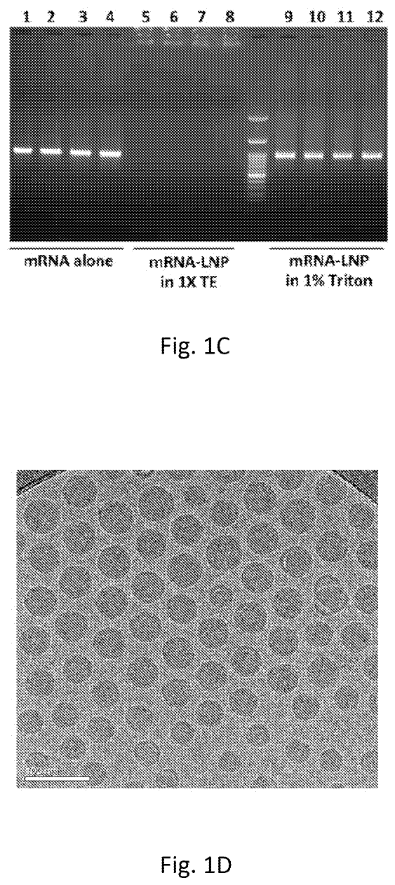

DNA templates were constructed to contain a T7 promoter site, a codon-optimized DENV2 E sequence (E₁₋₃₉₄-WT, E₁₋₃₉₄-N8R, E₁₋₃₉₄-W101G, and E₁₋₃₉₄-N8R-W101G), a 5′ UTR, IgG kappa leader sequence, a poly(A) tail region, and the alpha-globin gene 3′ UTR. Prior to IVT, the plasmid was linearized using EcoRV and purified with the NucleoSpin Gel and PCR Clean-up Kit (Macherey & Nagel Co. Düren, Germany). mRNA was synthesized according to the manufacturer's recommendations using HiScribe T7 (NEB, MA, USA) with co-transcriptional CleanCap® AG (Trilink, CA, USA) and N1-methyl-pseudouridine (Trilink, CA, USA). Synthesized mRNA was purified by DNase I (NEB, MA, USA) digestion followed by LiCl (Invitrogen, Thermo Fisher Scientific, Waltham, MA, USA) precipitation and 70% ethanol wash. Cellulose-based purification was performed to remove dsRNA from the transcribed mRNA. The final product was stored at −80° C. FIG. 1A depicts the process for the mRNA synthesis by IVT.

### 1.2 Site-Directed Mutagenesis

All point mutations of E₁₋₃₉₄-N8R, E₁₋₃₉₄-W101G, and E₁₋₃₉₄-N8R-W101G were generated using the QuikChange Kit (KAPA Biosystems), and the identities of the mutagenized products were verified by sequencing.

### 1.3 Virus Amplification for DENV2 16681

C6/36 cells were cultured in a medium consisting of 50% Mitsuhashi and Maramorosch (Sigma-Aldrich) plus 50% Dulbecco's modified Eagle's medium (DMEM, Gibco) supplemented with 10% fetal bovine serum (FBS, Gibco), 100 U/ml penicillin, 100 μg/ml streptomycin, and 0.25 μg/ml amphotericin B (Antibiotic-Antimycotic, Gibco). The C6/36 cells were infected with DENV2 16881 at MOI=1 and incubated at 28° C. for 7 days. The viruses were harvested from supernatant and propagated in the baby hamster kidney fibroblast (BHK-21) cell line. The infectious viral titers were quantified using plaque tests.

1.4. Preparation of mRNA-LNP

LNP formulations were prepared using a previously described method (Hsu et al., 2022). Briefly, four types of lipids were solubilized in ethanol:D-Lin-MC3-DMA (MedChemExpress, NJ, USA), DSPC (Avanti Polar Lipids, NY, USA), cholesterol (Sigma, MA, USA) and DMG-PEG 2000 (MedChemExpress, NJ, USA); the lipids were then mixed with a molar ratio of 50:10:38.5:1.5. The lipid mixture was combined with an aqueous sodium acetate buffer (25 mM, pH 4.5) containing mRNA at a flow rate ratio of 1:3 using NanoAssemblr® IGNITE NxGen Cartridges (Precision NanoSystems Inc., BC, Canada). LNP-encapsulated mRNA samples were dialyzed against PBS (pH 7.4) at 4° C. Then, the mRNA-LNP were concentrated using Amicon Ultra Centrifugal Filters (10 K. MWCO; Millipore, Burlington, MA, USA) and passed through a 0.45-mm filter. FIG. 1B depicts the process for the mRNA-LNP synthesis using a microfluidic device.

1.5. Characterization of mRNA-LNP

The particle size distribution, polydispersity index (PDI) value, and zeta potential of DENV2 E mRNA-LNP were analyzed by dynamic light scattering (DLS, Zetasizer Nano ZS, Malvern Instruments, UK). The sample was diluted 100-fold and equilibrated for 120 seconds at 25° C. prior to size and zeta potential measurements. The hydrodynamic diameter (z-average) and zeta potential of DENV2 E mRNA-LNP were analyzed by Zetasizer software, version 7.11 (www.malvern.com). The morphology of DENV2 E mRNA-LNP was observed in a dry state using cryogenic transmission electron microscopy (cryo-TEM, Tecnai F20, Philips, Eindhoven, the Netherlands). Briefly, the sample solution was diluted 10-fold and transferred onto a 300-mesh copper grid covered with porous carbon film (HC300-Cu, PELCO) before blotting and plunging in a 100% humidity temperature-controlled chamber by Vitroblot (FEI). The copper grids were stored under liquid nitrogen and transferred to the electron microscope on a cryo-stage for imaging. The mRNA encapsulation efficiency (EE %) and the concentration were determined by using the Quant-iT RiboGreen RNA assay kit (Invitrogen, Thermo Fisher Scientific, Waltham, MA, USA). The mRNA integrity of free mRNA and LNP-mRNA was analyzed by an agarose gel retardation assay. LNP-mRNA complexes were solubilized with 1% Triton X-100, and the integrity of released mRNA was inspected by agarose gel.

1.6. In Vitro Protein Expression of DENV2 E mRNA-LNP by Flow Cytometry

DENV2 E mRNA-LNP (E₁₋₃₉₄-WT, E₁₋₃₉₄-N8R, E₁₋₃₉₄-W101G, and E₁₋₃₉₄-N8R-W101G) were individually transfected into 293T cells and cultured at 37° C. in DMEM medium containing 10% FBS for 24 hours. Then, the cells were collected and centrifuged. The cell pellet was washed with PBS via centrifugation and followed by incubation with fixation and permeabilization solution (BD, catalog no. 554714) for 20 min at 4° C. Cells were then stained with 1 ng/ml DENV-specific monoclonal antibodies (4G2, DB32-6, and DB39-2) for 1 hour at 4° C., followed by incubation with PE-conjugated goat anti-mouse IgG (1:500) for 1 hour at 4° C. The samples were detected by flow cytometer and a minimum of 1×10⁴ events for each sample were recorded and analyzed with FlowJo software.

### 1.7. Mouse Immunization

All procedures involving animal studies were approved and performed in accordance with guidelines set by the Institutional Animal Care and Use Committee (IACUC) at Academia Sinica, Taiwan. Groups of 6-to-8-week-old BALB/c mice were immunized via intramuscular injection with 10 μg of DENV2 E mRNA-LNP or control solution (saline) at weeks 0, 2, and 4. Serum samples were collected 6 weeks after the first immunization and stored at −80° C. until further use.

### 1.8. ELISA

Measurements of binding antibodies in sera of mice immunized by DENV2 E mRNA-LNP were made by ELISA. Briefly, ELISA plates were coated with 0.5 ng/ml of DENV1-4 recombinant E proteins (PROSPEC) and incubated at 4° C. overnight, followed by blocking with PBS containing 1% bovine serum albumin (BSA) at RT for 2 hours. After blocking, the wells were washed twice with PBS. Then, sera that were serially diluted with 1% BSA in PBS were added into each well in triplicate, and the plate was incubated at RT for 1 hour. The plates were washed three times with PBS containing 0.1% Tween-20 (PBST0.1) and then incubated for 1 hour with peroxidase-affinipure goat anti-mouse IgG (H+L) (Jackson ImmunoResearch) (1:5000 dilution). After three washes with PBST0.1, the signal was produced using 3,3′5,5′-Tetramethylbenzidine (TMB) color development (TMBW-1000-01, SURMODICS). Finally, the reaction was stopped with 3 N HCl, and absorbance was measured at 450 nm by an ELISA reader (Versa Max Tunable Microplate Reader; Molecular Devices).

### 1.9. Plaque Reduction Neutralization Titer (PRNT) Assay

Sera from animals injected with DENV2 E mRNA-LNPs were serially diluted in PBS and pre-incubated with 100 plaque-forming units (PFU) DENV2 for 1 hour at 37° C. The mixtures were then added to pre-seeded BHK-21 cells for 1 hour at 37° C. The virus-containing culture medium was removed and replaced with DMEM containing 2% FBS and 1% methyl-cellulose for an additional 4-day incubation. The cells were fixed with 10% formaldehyde overnight and stained with 0.5% crystal violet for 20 min. The plates were then washed with tap water, and plaque numbers formed at each dilution were counted. Each experiment was performed in triplicate. Plaque reduction was calculated as follows: Inhibition percentage=100×[1−(plaque number incubated with immunized serum/plaque number without immunized serum)]. The 50% plaque reduction (PRNT₅₀) value was calculated with Prism software. The DENV2 strain 16881 was used in this study.

### 1.10. Antibody-Dependent Enhancement (ADE) Assay

Mouse serum samples were serially diluted in serum-free DMEM medium and incubated with DENV2 16881 (MOI=0.2) for 1 hour at 37° C. The serum-virus mixtures were then mixed with 3×10⁴ K562 cells and incubated for 2 hours at 37° C. The cells were then incubated with 2% FBS in DMEM at 37° C. for 4 days. The serially diluted supernatant was collected from K562 cells, and the PRNT assay was performed to detect virus activity.

### 2. Results

2.1. Physiochemical Characterization of mRNA-LNP Complex

The integrity of mRNA packaging into LNP was characterized by an agarose gel electrophoresis assay. FIG. 1C shows that intact mRNA-LNP complexes were trapped within the gel well (lanes 5-8). However, pre-treatment of LNP-mRNA complexes with 1% Triton X-100 (lanes 9-12) yielded bands at the same molecular weight as the naked mRNAs (lanes 1-4). The data suggest that the mRNAs were successfully packed into LNPs, and disrupting the LNPs could release the mRNA without causing degradation or otherwise disrupting mRNA integrity.

Measurements of the average particle sizes of the mRNA-LNP complexes were between 80-110 nm, and zeta potentials ranged between +8 to +12.5 mV according to dynamic light scattering (DLS) experiments. Most importantly, the mRNA-LNP all had uniform distributions in aqueous solution without aggregation, as indicated by their PDI values being lower than 0.3 (Table 1).

TABLE 1
shows the molar ratio and physicochemical
parameters of the mRNA-LNP complex.

| | | | | E₁₋₃₉₄-N8R- |
|---|---|---|---|---|
| | E₁₋₃₉₄-WT | B₁₋₃₉₄-N8R | E₁₋₃₉₄-W101G | W101G |
| Ionizable lipid | D-Lin-MC3- | D-Lin-MC3- | D-Lin-MC3- | D-Lin-MC3- |
| | DMA | DMA | DMA | DMA |
| Lipid molar ratio (%) | 50:10:38.5:1.5 | 50:10:38.5:1.5 | 50:10:38.5:1.5 | 50:10:38.5:1.5 |
| (lonizable lipid: | | | | |
| DSPC:Cholesterol: | | | | |
| PEG) | | | | |
| Molar N/P | 8 | 8 | 8 | 8 |
| Size (nm) | 110 | 95 | 82.4 | 90.5 |
| PDI | 0.12 | 0.1 | 0.12 | 0.19 |
| Zeta potential (mV) | +12.5 | +8.1 | +9.3 | +12.2 |
| Encapsulation | 98% | 97% | 96% | 97% |
| efficiency (%) | | | | |
| Final Yield (%) | 67.3% | 62% | 66.5% | 71.1% |

Cryo-EM imaging of the mRNA-LNP confirmed the homogenous distribution of the complex and showed that the complexes were spherical (FIG. 1D).

### 2.2. Optimization of In-Vitro Protein Expression

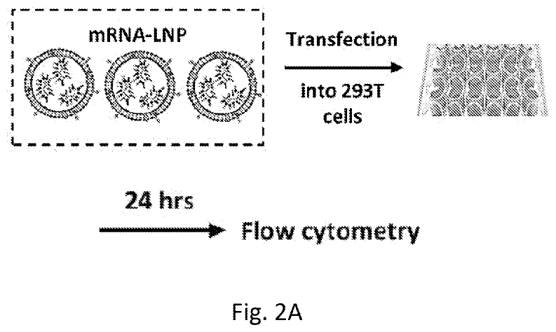

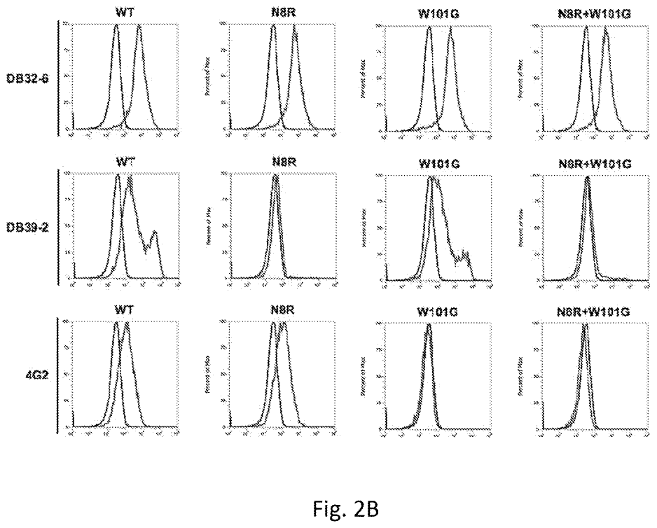

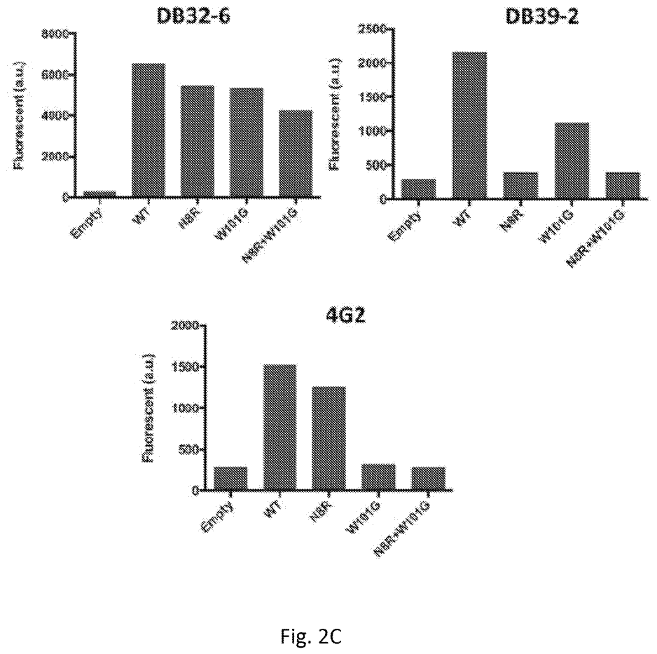

The four DENV2 E mRNA-LNPs, called E₁₋₃₉₄-WT, E₁₋₃₉₄-N8R, E₁₋₃₉₄-W101G and E₁₋₃₉₄-N8R-W101G, were individually transfected into 293T cells, as shown in FIG. 2A. Prior to flow cytometry analysis, the cells were collected and incubated with monoclonal antibodies DB32-6, DB39-2, and 4G2. DB32-6 and DB39-2 were purified in our lab previously (Tang et al., 2015); DB32-6 specifically binds EDIII of DENV2 (Tang et al., 2015), while DB39-2 binds N8 EDI of DENV1-4 (Tang et al., 2015). In contrast, 4G2 binds to the highly conserved amino acids of domain II for DENV1-4 (Rajamanonmani et al., 2009). The DB32-6 antibody was used to verify that the mutations indeed caused structural changes to the E protein.

First, the success of mRNA transfection was evaluated by flow cytometry. Analysis with monoclonal antibody DB32-6 showed successful expression of the DENV2 E protein after transfection with WT and mutant mRNA-LNP. However, DB39-2 and 4G2 did not produce respective signals for E₁₋₃₉₄-N8R and E₁₋₃₉₄-W101G mutant mRNA-LNP-treated cells. Moreover, transfection with E₁₋₃₉₄-N8R-W101G mRNA-LNP did not generate signal from either the DB39-2 or 4G2 monoclonal antibody probes. Together, these data suggest that our mutant mRNA-LNP produce proteins with disrupted structures, as expected (FIGS. 2B and 2C). Table 2 shows the flow cytometry data against specific antibodies; (+) represents the detection of protein with the antibody, while (−) represents no detection.

TABLE 2

| | Antibodies | | |
|---|---|---|---|
| | DB32-6 | DB392- | 4G2 |
| E₁₋₃₉₄-WT | + | + | + |
| E₁₋₃₉₄-N8R | + | − | + |
| E₁₋₃₉₄-W101G | + | + | − |
| B₁₋₃₉₄-N8R-W101G | + | − | − |

2.3. Modified mRNA-LNP Vaccine Elicits an Antigen-Specific Immune Response in Mice Model

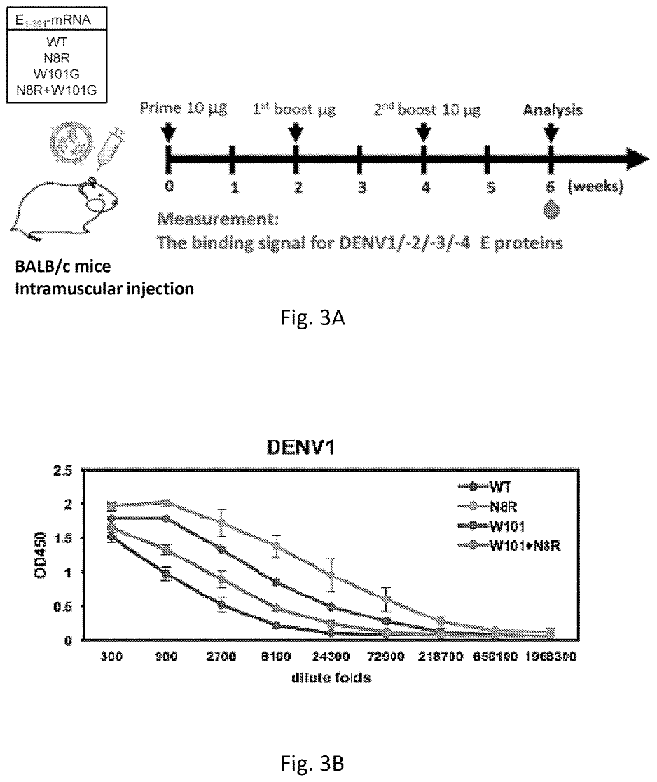

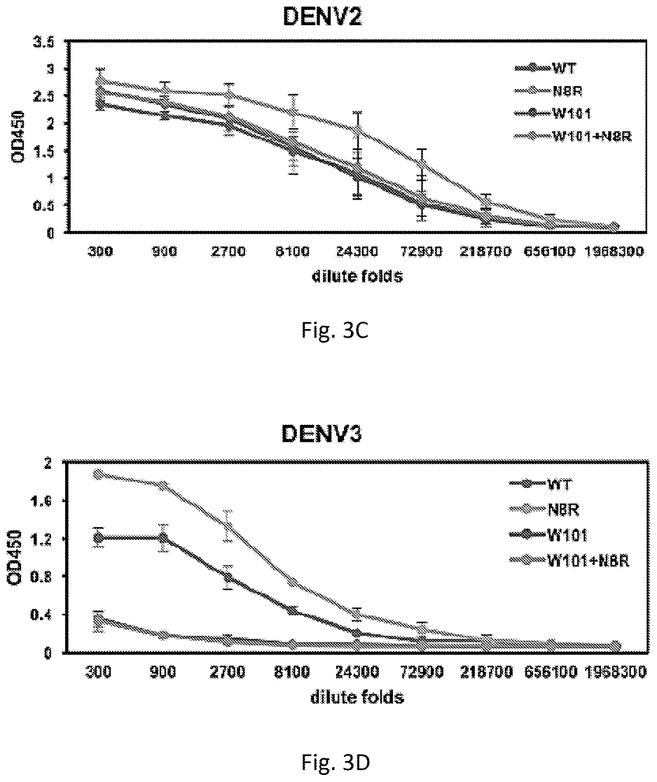

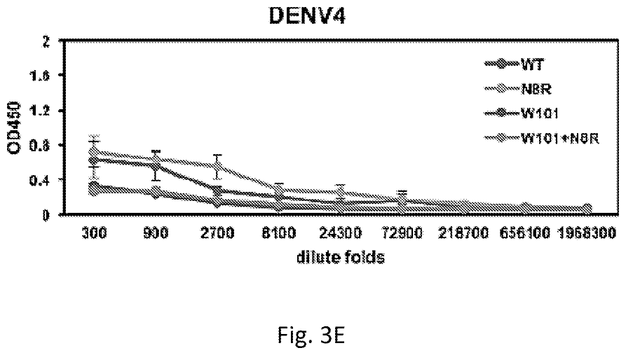

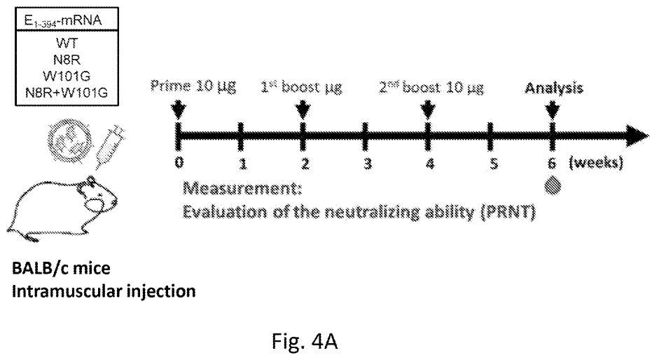

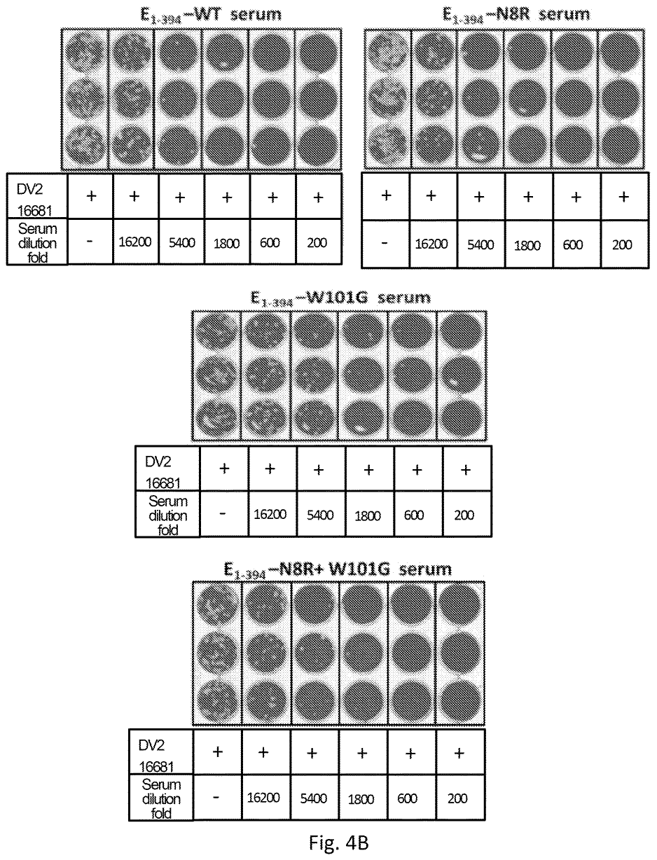

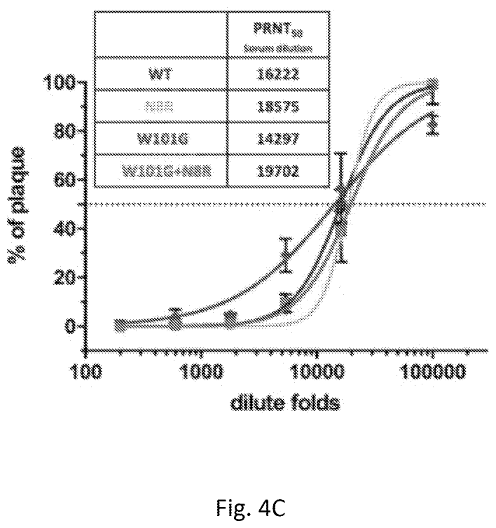

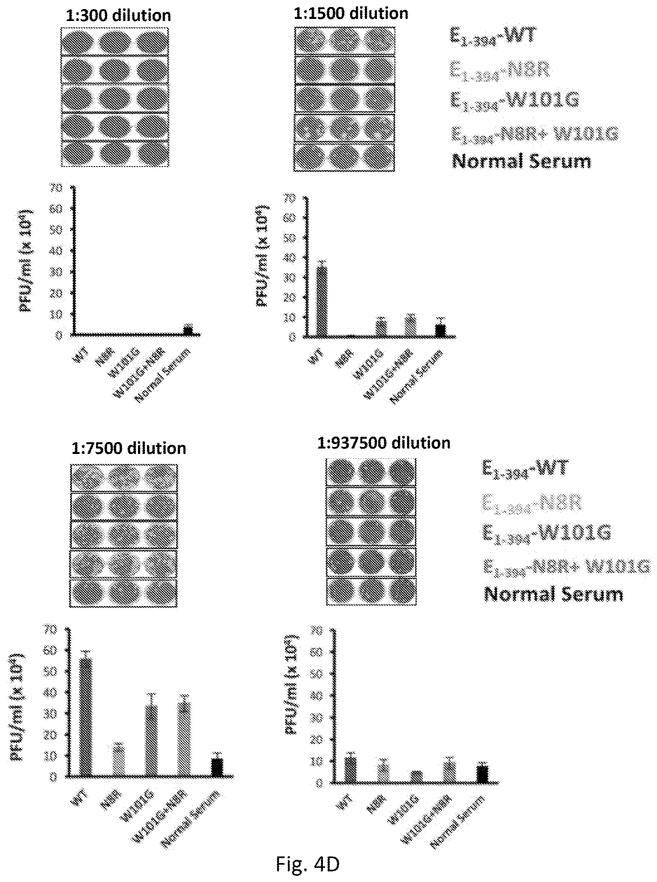

To test the immunogenicity of the mRNA-LNP, 8-week-old BALB/c mice were vaccinated with a series of intramuscular (i.m.) injections of 10 μg of mRNA per dose at two-week intervals (FIG. 3A and FIG. 4A). Two weeks after the last immunization, serum samples were collected to evaluate the neutralizing antibody binding activity toward all four DENV serotypes by ELISA. The neutralizing activity of DENV antibodies was evaluated by performing the PRNT assay in BHK-21 cells. The optimal working concentration of DENV2 for the PRNT assay was 1 MOI (multiplicity of infection).

The neutralizing antibody binding activity in serial dilutions of sera were detected by ELISA screening against DENV1-4 serotypes (FIGS. 3B to 3B). Sera of mice inoculated with the E₁₋₃₉₄-N8R-mRNA-LNP exhibited high binding activity against DENV1-3 serotypes but showed minimal binding with DENV4 (FIGS. 3B to 3E). Table 3 shows the ELISA data for each antibody; the signal level is represented as follows, in descending order: +++>++>+>−.

Binding signal (OD₄₅₀)
E protein

| | DV-1 | DV-2 | DV-3 | DV-4 |
|---|---|---|---|---|
| WT | +++ | +++ | ++ | + |
| N8R | +++ | +++ | +++ | + |
| W101G | +++ | +++ | − | − |
| N8R-W101G | ++ | +++ | − | − |

Interestingly, the sera of mice injected with double mutant (E₁₋₃₉₄-N8R-W101G-mRNA-LNP) had low binding activities for both DENV3 and 4 serotypes and highest neutralizing antibody levels against DENV2, with PRNT₅₀ values around 19,702 (FIG. 3B to 3E, FIG. 4B, and FIG. 4C). Notably, mice immunized with E₁₋₃₉₄-N8R-mRNA-LNP showed cross-neutralization with nearly all DENV serotypes and had the second highest neutralizing antibody response against DENV2, with a PRNT₅₀ value around 18,575.

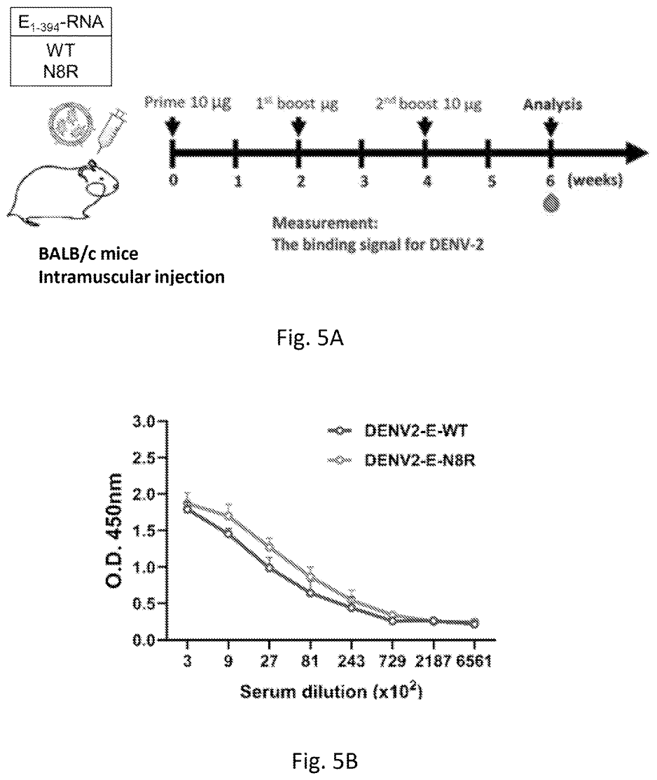

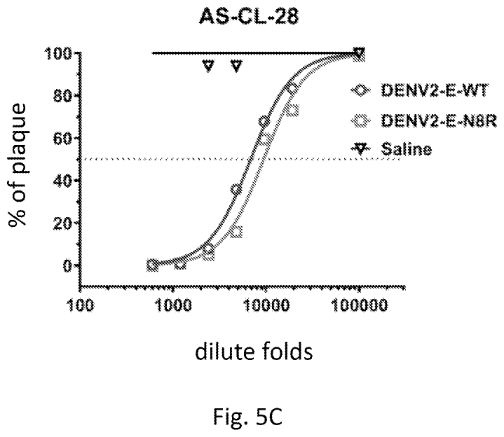

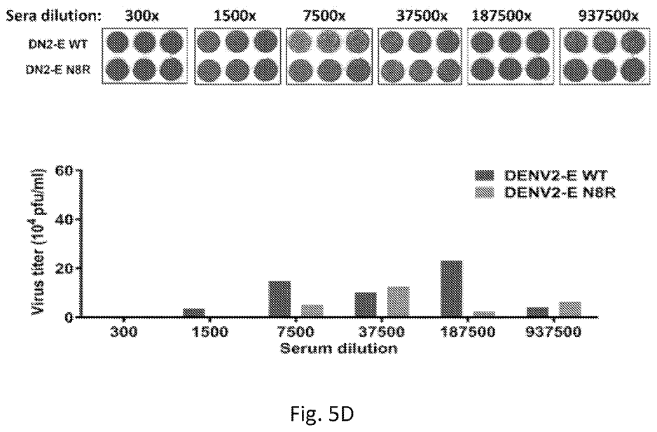

In further experiments, we prepared E1394-N8R-mRNA-LNP and E₁₋₃₉₄-WT-mRNA-LNP using another ionizable lipid (AS-CL-28). These formulations were tested for their NSR immunization potential. BALB/c mice were immunized with 10 g of the mRNA-LNP complex by intramuscular injection (FIG. 5A). The sera from mice immunized with E₁₋₃₉₄-N8R-mRNA-LNP showed high binding activity against the DENV2 serotype, as well as higher neutralizing antibody levels (FIG. 5B) and reduced the ADE burden (FIG. 5C) compared to sera from E₁₋₃₉₄-WT-mRNA-LNP-immunized animals. Of note, E1-394-N8R-mRNA-LNP (prepared with AS-CL-28 ionizable lipid) caused lower levels of viral replication than E1-394-WT-mRNA-LNP at all tested serial dilutions (FIG. 5D). Together these data support the idea that mRNA-LNP with N8R mutation induce high neutralizing antibody titers and minimal ADE response against DENV2.

### 2.4. In Vitro ADE of Viral Replication Activity by Immunized Mouse Sera

To evaluate whether the mutations in EDI and EDII could attenuate the ADE response, an infection assay was performed using Fcγ receptor-positive K562 cells. K562 cells can be infected by DENV in the presence of enhancing antibodies via Fc-mediated endocytosis (Messer et al., 2014). DENV2 virus and serially diluted sera from vaccinated mice were co-incubated with K562 cells for 5 days; K562 cells infected by DENV2 in the presence of diluted sera from naïve mice served as a negative control. After the incubation period, supernatants from K562 cells were collected for viral replication measurements. The levels of infectious virus in the supernatants of K562 cells treated with immune sera were determined by performing the PRNT assay with BHK21 cells. Sera from E₁₋₃₉₄-N8R-mRNA-LNP-vaccinated mice caused diminished viral replication relative to the other mutant types. The dilution of 1:7500 exhibited the lowest ADE response and best protection ability compared to any other condition (FIG. 4D). Together these data support the idea that mRNA-LNP with N8R mutation induce high neutralizing antibody titers and minimal ADE response against DENV2.

In addition, mRNA-LNP prepared using another cationic lipid (AS-CL-28) exhibited a similar trend, with E₁₋₃₉₄-N8R-immunized mice displaying higher levels of neutralizing antibodies and reduced in vivo and in vitro ADE potential, as compared to E₁₋₃₉₄-WT-immunized mice. Further, our suckling mice experiments revealed that sera from E₁₋₃₉₄-N8R-immunized mice could mediate robust neutralization and protection against DENV2 infection, and these effects were stronger than those of E₁₋₃₉₄-WT-immunized sera.

### 2.5 Survival Test

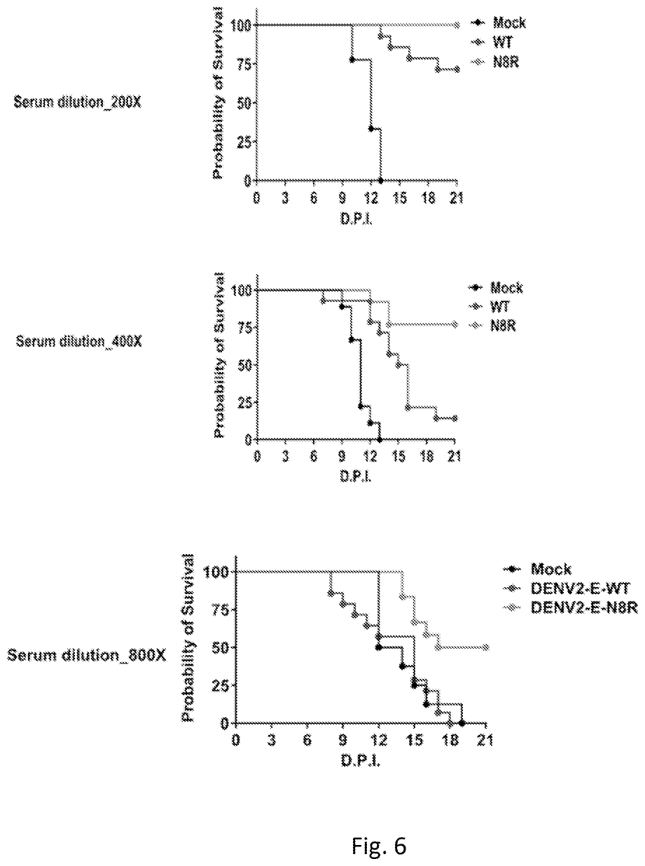

The protective efficacy of neutralizing antibodies in collected sera was evaluated in 2-day-old ICR suckling mice. Sera from BALB/c mice immunized with E₁₋₃₉₄-N8R-mRNA-LNP, E₁₋₃₉₄-WT-mRNA-LNP, and PBS (mock) were serially diluted and incubated with DENV2 virus before intracranial administration to the suckling mice. Survival rates were monitored for 28 days after injection. As shown in FIG. 6, suckling mice from the E₁₋₃₉₄-N8R-mRNA-LNP group showed a 100% survival rate at the dose of 1:200 diluted sera. Moreover, other sera dilutions (e.g., 1:400 and 1:800) showed higher protection for the E₁₋₃₉₄-N8R-mRNA-LNP group than the E₁₋₃₉₄-WT-mRNA-LNP group. Compared to sera from mock-infected mice, 1:200 and 1:400 diluted sera from E₁₋₃₉₄-WT-mRNA-LNP groups exhibited better protection. Overall, the results consistently showed that E₁₋₃₉₄-N8R-mRNA-LNP-immunized sera effectively neutralized the DENV2 virus and better protected suckling mice than E₁₋₃₉₄-WT-mRNA-LNP-immunized sera (FIG. 6).

### 3. Discussion

With the continued global spread of DENV infection and its attendant morbidity particularly in tropical and subtropical regions (Khetarpal and Khanna, 2016), there is a need to develop effective vaccines and therapeutics. Currently, only one vaccine, Dengvaxia, is licensed in most dengue-prone areas (Tully and Griffiths, 2021), though many vaccines are currently under evaluation (Torres-Flores et al., 2022). Recently, mRNA vaccines have garnered significant attention for their potential to prevent infections by flaviviruses (August et al., 2021; Ge et al., 2022; VanBlargan et al., 2018), including DENV (Wollner and Richner, 2021). A major advantage of the mRNA platform is that it allows for quick design and screening of target antigens. Moreover, the encapsulation of modified mRNAs into LNP protects mRNAs from external factors that would otherwise cause degradation (Chaudhary et al., 2021). In our study, we designed mutant mRNA sequences with N8R, W101G and N8R-W101G substitutions. These mutations appear to reduce the ADE response. We verified that the mutant mRNA sequences have expected binding profiles with three different antibodies (FIGS. 2A to 2C). Previously, an N8R-mutated DNA vaccine was characterized and shown to elicit better neutralizing antibody response and reduce the potential for ADE compared to the WT sequence (Tang et al., 2015). However, appropriate technology to support successful delivery of DNA for immunization is lacking, and the vaccine showed overall low antibody titer. Therefore, we altered our approach to utilize mRNA-LNP immunization, which increased the antibody titer by about 10-fold.

Dengue virus strains are categorized as four different serotypes (DENV1-4) with amino acid sequence variations up to 35% (Wollner et al., 2021). Therefore, the vaccines developed for one serotype might not be effective against heterologous serotypes. Generally, dengue-affected areas have cases from all serotypes (Guzman et al., 2010), so most DENV vaccines under clinical evaluation are designed to target sequences from all four serotypes (Pinheiro-Michelsen et al., 2020). Nevertheless, there remains an unsatisfied need for a successful DENV vaccine that exhibits low ADE responses for all four serotypes. To address this problem, we have identified and substituted key epitopes that affect both titer of neutralizing antibodies and risk of ADE. Immunization of mice with N8R-mRNA-LNP produced high levels of neutralizing antibodies against DENV2, and minimal levels against DENV1>DENV3>DENV4. In comparison with the WT vaccine, the N8R mutant had higher levels of neutralizing antibodies for all four serotypes. Recently, Wollner et al., designed an mRNA-LNP vaccine encoding DENV1 prM/E protein, and this mRNA induced high levels of neutralizing antibodies against DENV1 with minimal ADE response against DENV1 and 2 (Wollner et al., 2021). In another study, researchers utilized DENV2-encoding mRNA vaccines and found that mRNA that generates proteins with the enveloped structure induces high neutralizing antibody titer and minimal ADE response against DENV2 (Zhang et al., 2020). To overcome low neutralizing antibody titer against all four serotypes, He et al. has proposed two mRNA constructs. The first consists of EDIII from DENV1 and NS1 from DENV2, while the other has mRNA for EDIII from DENV4 and NS1 from DENV3. The sera from mice immunized with a combination of both mRNA constructs showed high neutralizing antibody titers against all serotypes (He et al., 2022). However, this design would add a complication of needing to balance neutralizing antibody titers in clinical trials. Moreover, we found that E₁₋₃₉₄-N8R mRNA-LNP exhibited higher neutralizing antibody titers (PRNT₅₀=18585; log 10=4.26) than previously published DENV mRNA-LNP complexes (He et al., 2022, Zhang et al., 2020). Therefore, we sought to design a vaccine with balanced protective efficacy against all four serotypes.

Protection efficacy is expected to be directly proportional to the antibody titer induced after vaccination. In our study, immunization with the N8R mutant induced high neutralizing antibody titers against all four serotypes. According to the PRNT assay, mRNA-LNP with N8R, W101G, and N8R-W101G mutants respectively induced average endpoint dilution titers of 18,575, 14,297, and 19,702 (FIG. 4C). Interestingly, the N8R-W101-mRNA-LNP induced high titers of antibodies against DENV2, but it was not particularly effective against other serotypes (FIGS. 3B to 3E). This result highlights the fact that a vaccine developed against one serotype may or may not be effective against another. In light of these findings, we considered the parameters that influence neutralizing antibody titer for all four serotypes. The high binding of DENV2 E mRNA-LNP immunized sera with DENV1-3 suggests that our approach is promising for the design of a safer vaccine with minimal ADE response. Since vaccines against the E protein may cause an ADE response, we modified the E protein structure. By replacing asparagine with arginine in the 8th position, the protective efficacy against DENV was enhanced, and the ADE response to DENV2 was also reduced. Thus, we can conclude that the mutations we introduced into the E protein reduced the ADE responses compared with WT. Immune sera from E₁₋₃₉₄-WT-vaccinated mice showed ADE effects against DENV2 and also had relatively low levels of neutralizing antibodies. Thus, the N8R-mRNA-LNP vaccine not only had reduced ADE response, but it also caused enhanced production of neutralizing antibodies against DENV1-3 serotypes; highly diluted sera showed high protection efficiency when compared with other mRNA types (FIG. 4D). Altogether, these findings lead us to conclude that the N8R mutation not only enhances neutralizing antibody response but also reduces the potential for ADE responses.

Overall, our study introduces a potentially effective mRNA vaccine encoding E protein with a specific mutation, and it contributes to our understanding of the role this mutation plays in ADE. Thus, the current study lays essential groundwork and provides critical insights that may aid in the development of successful mRNA vaccines against DENV.

### Sequence Information

### Amino Acid Sequence of Wild Type E Protein of Dengue Virus Serotype 1 (DENV1) (394 a.a) (SEQ ID NO: 1)

Amino acid sequence of wild type E protein of dengue virus serotype 1
(DENV1) (394 a.a)
(SEQ ID NO: 1)
MRCVGIGNRDFVEGLSGATWVDVVLEHGSCVTTMAKDKPTLDIELLKTEVTNPAVLRKLCIEAKI
SNTTTDSRCPTQGEATLVEEQDTNFVCRRTFVDRGWGNGCGLFGKGSLITCAKFKCVTKLEGKIV
QYENLKYSVIVTVHTGDQHQVGNETTEHGTTATITPQAPTSEIQLTDYGALTLDCSPRTGLDFNE
MVLLTMKEKSWLVHKQWFLDLPLPWTSGASTSQETWNRQDLLVTFKTAHAKKQEVVVLGSQEGAM
HTALTGATEIQTSGTTTIFAGHLKCRLKMDKLTLKGVSYVMCTGSFKLEKEVAETQHGTVLVQVK
YEGTDAPCKIPFSSQDEKGVTQNGRLITANPIVTDKEKPVNIEAEPPFGESYLVVGAGEKALKLS
WFKK
Amino acid sequence of wild type E protein of dengue virus serotype 2
(DENV2) (394 a.a)
(SEQ ID NO: 2)
MRCIGMSNRDFVEGVSGGSWVDIVLEHGSCVTTMAKNKPTLDFELIKTEAKQPATLRKYCIEAKL
TNTTTESRCPTQGEPSLNEEQDKRFVCKHSMVDRGWGNGCGLFGKGGIVTCAMFRCKKNMEGKVV
QPENLEYTIVITPHSGEEHAVGNDTGKHGKEIKITPQSSITEAELTGYGTVTMECSPRTGLDFNE
MVLLQMENKAWLVHRQWFLDLPLPWLPGADTQGSNWIQKETLVTFKNPHAKKQDVVVLGSQEGAM
HTALTGATEIQMSSGNLLFTGHLKCRLRMDKLQLKGMSYSMCTGKFKVVKEIAETQHGTIVIRVQ
YEGDGSPCKIPFEIMDLEKRHVLGRLITVNPIVTEKDSPVNIEAEPPFGDSYIIIGVEPGQLKLN
WFKK
Amino acid sequence of wild type E protein of dengue virus serotype 3
(DENV3) (392 a.a)
(SEQ ID NO: 3)
MRCVGVGNRDFVEGLSGATWVDVVLEHGGCVTTMAKNKPTLDIELQKTEATQLATLRKLCIEGKI
TNITTDSRCPTQGEATLPEEQDQNYVCKHTYVDRGWGNGCGLFGKGSLVTCAKFQCLEPIEGKVV
QYENLKYTVIITVHTGDQHQVGNETQGVTAEITPQASTTEAILPEYGTLGLECSPRTGLDFNEMI
LLTMKNKAWMVHRQWFFDLPLPWTSGATTETPTWNRKELLVTFKNAHAKKQEVVVLGSQEGAMHT
ALTGATEIQNSGGTSIFAGHLKCRLKMDKLELKGMSYAMCTNTFVLKKEVSETQHGTILIKVEYK
GEDVPCKIPFSTEDGQGKAHNGRLITANPVVTKKEEPVNIEAEPPFGESNIVIGIGDNALKINWY
KK
Amino acid sequence of wild type E protein of dengue virus serotype 4
(DENV4) (394 a.a)
(SEQ ID NO: 4)
MRCVGVGNRDFVEGVSGGAWVDLVLEHGGCVTTMAQGKPTLDFELTKTTAKEVALLRTYCIEASI
SNITTATRCPTQGEPYLKEEQDQQYICRRDVVDRGWGNGCGLFGKGGVVTCAKFLCSGKITGNLV
QIENLEYTVVVTVHNGDTPAVGNDTSHHGVTATITPRSPSVEVKLPDYGELTLDCEPRSGIDFIE
MILMKMKKKTWLVHKQWFLDLPLPWTAGADTSEVHWNHKERMVTFKVPHAKRQDVTVLGSQEGAM
HSALAGATEVDSGDGNHMFAGHLKCKVRMEKLRIKGMSYTMCSGKFSIDKEMAETQHGTAVVKVK
YEGAGAPCKVPIEIRDVNKEKVVGRIISSTPFAENTNSVTNIELEPPFGDSYIVIGVGESALTLH
WFRK
Amino acid sequence of mutant E protein N8R of dengue virus serotype 2
(DENV2) (394 a.a)
(SEQ ID NO: 5)
MRCIGMSRRDFVEGVSGGSWVDIVLEHGSCVTTMAKNKPTLDFELIKTEAKQPATLRKYCIEAKL
TNTTTESRCPTQGEPSLNEEQDKRFVCKHSMVDRGWGNGCGLFGKGGIVTCAMFRCKKNMEGKVV
QPENLEYTIVITPHSGEEHAVGNDTGKHGKEIKITPQSSITEAELTGYGTVTMECSPRTGLDFNE
MVLLQMENKAWLVHRQWFLDLPLPWLPGADTQGSNWIQKETLVTFKNPHAKKQDVVVLGSQEGAM
HTALTGATEIQMSSGNLLFTGHLKCRLRMDKLQLKGMSYSMCTGKFKVVKEIAETQHGTIVIRVQ
YEGDGSPCKIPFEIMDLEKRHVLGRLITVNPIVTEKDSPVNIEAEPPFGDSYIIIGVEPGQLKLN
WFKK
Amino acid sequence of mutant E protein W101G of dengue virus serotype 2
(DENV2) (394 a.a)
(SEQ ID NO: 6)
MRCIGMSNRDFVEGVSGGSWVDIVLEHGSCVTTMAKNKPTLDFELIKTEAKQPATLRKYCIEAKL
TNTTTESRCPTQGEPSLNEEQDKRFVCKHSMVDRGGGNGCGLFGKGGIVTCAMERCKKNMEGKVV
QPENLEYTIVITPHSGEEHAVGNDTGKHGKEIKITPQSSITEAELTGYGTVTMECSPRTGLDFNE
MVLLQMENKAWLVHRQWFLDLPLPWLPGADTQGSNWIQKETLVTFKNPHAKKQDVVVLGSQEGAM
HTALTGATEIQMSSGNLLFTGHLKCRLRMDKLQLKGMSYSMCTGKFKVVKEIAETQHGTIVIRVQ
YEGDGSPCKIPFEIMDLEKRHVLGRLITVNPIVTEKDSPVNIEAEPPFGDSYIIIGVEPGQLKLN
WFKK
Amino acid sequence of mutant E protein N8R + W101G of dengue
virus serotype 2 (DENV2)
(394 a.a)
(SEQ ID NO: 7)
MRCIGMSRRDFVEGVSGGSWVDIVLEHGSCVTTMAKNKPTLDFELIKTEAKQPATLRKYCIEAKL
TNTTTESRCPTQGEPSLNEEQDKRFVCKHSMVDRGGGNGCGLFGKGGIVTCAMERCKKNMEGKVV
QPENLEYTIVITPHSGEEHAVGNDTGKHGKEIKITPQSSITEAELTGYGTVTMECSPRTGLDFNE
MVLLQMENKAWLVHRQWFLDLPLPWLPGADTQGSNWIQKETLVTFKNPHAKKQDVVVLGSQEGAM
HTALTGATEIQMSSGNLLFTGHLKCRLRMDKLQLKGMSYSMCTGKFKVVKEIAETQHGTIVIRVQ
YEGDGSPCKIPFEIMDLEKRHVLGRLITVNPIVTEKDSPVNIEAEPPFGDSYIIIGVEPGQLKLN
WFKK
DNA sequence of mutant E protein NSR of dengue virus serotype 2
(DENV2)
(SEQ ID NO: 8)
Atgaggtgtattggaatgtctagacgggattttgtggagggagtctctggcgggagttgggtgga
tattgtcctggagcacggaagctgcgtgaccacaatggccaagaacaagcccaccctggacttcg
agctgatcaagaccgaggccaagcagcctgccacactgcggaagtactgcatcgaggccaagctg
accaacaccacaaccgagagcagatgtcccacacagggcgagccttccctgaatgaggagcagga
caagcggttcgtgtgcaagcactctatggtggataggggatggggaaacggatgtggcctgttcg
gcaagggcggcatcgtgacctgcgccatgtttcggtgtaagaagaacatggagggcaaggtggtg
cagcctgagaatctggagtacaccatcgtgatcacaccacacagcggagaggagcacgcagtggg
aaatgacaccggcaagcacggcaaggagatcaagatcacaccacagagctccatcaccgaggccg
agctgacaggctatggcacagtgaccatggagtgctccccaaggaccggcctggatttcaacgag
atggtgctgctgcagatggagaataaggcctggctggtgcaccgccagtggtttctggacctgcc
actgccatggctgcctggagcagatacccagggctccaactggatccagaaggagacactggtga
ccttcaagaatccacacgccaagaagcaggacgtggtggtgctgggctctcaggagggagcaatg
cacacagccctgaccggagccacagagatccagatgtctagcggcaatctgctgttcaccggcca
cctgaagtgccggctgagaatggataagctgcagctgaagggcatgagctactccatgtgcaccg
gcaagtttaaggtggtgaaggagatcgccgagacacagcacggcacaatcgtgatcagggtgcag
tatgagggcgacggcagcccttgtaagatcccattcgagatcatggatctggagaagcggcacgt
gctgggcagactgatcaccgtgaacccaatcgtgacagagaaggactctcccgtgaatatcgagg
ccgagcccccttttggcgatagctacatcatcatcggcgtggagcccggccagctgaagctgaac
tggttcaagaagtaa
DNA sequence of mutant E protein W101G
(SEQ ID NO: 9)
Atgaggtgtattggaatgtctaaccgggattttgtggagggagtctctggcgggagttgggtgga
tattgtcctggagcacggaagctgcgtgaccacaatggccaagaacaagcccaccctggacttcg
agctgatcaagaccgaggccaagcagcctgccacactgcggaagtactgcatcgaggccaagctg
accaacaccacaaccgagagcagatgtcccacacagggcgagccttccctgaatgaggagcagga
caagcggttcgtgtgcaagcactctatggtggataggggaggaggaaacggatgtggcctgttcg
gcaagggcggcatcgtgacctgcgccatgtttcggtgtaagaagaacatggagggcaaggtggtg
cagcctgagaatctggagtacaccatcgtgatcacaccacacagcggagaggagcacgcagtggg
aaatgacaccggcaagcacggcaaggagatcaagatcacaccacagagctccatcaccgaggccg
agctgacaggctatggcacagtgaccatggagtgctccccaaggaccggcctggatttcaacgag
atggtgctgctgcagatggagaataaggcctggctggtgcaccgccagtggfttctggacctgcc
actgccatggctgcctggagcagatacccagggctccaactggatccagaaggagacactggtga
ccttcaagaatccacacgccaagaagcaggacgtggtggtgctgggctctcaggagggagcaatg
cacacagccctgaccggagccacagagatccagatgtctagcggcaatctgctgttcaccggcca
cctgaagtgccggctgagaatggataagctgcagctgaagggcatgagctactccatgtgcaccg
gcaagtttaaggtggtgaaggagatcgccgagacacagcacggcacaatcgtgatcagggtgcag
tatgagggcgacggcagcccttgtaagatcccattcgagatcatggatctggagaagcggcacgt
gctgggcagactgatcaccgtgaacccaatcgtgacagagaaggactctcccgtgaatatcgagg
ccgagcccccttttggcgatagctacatcatcatcggcgtggagcccggccagctgaagctgaac
tggttcaagaagtaa
DNA sequence of mutant E protein N8R + W101G
(SEQ ID NO: 10)
Atgaggtgtattggaatgtctagacgggattttgtggagggagtctctggcgggagttgggtgga
tattgtcctggagcacggaagctgcgtgaccacaatggccaagaacaagcccaccctggacttcg
agctgatcaagaccgaggccaagcagcctgccacactgcggaagtactgcatcgaggccaagctg
accaacaccacaaccgagagcagatgtcccacacagggcgagccttccctgaatgaggagcagga
caagcggttcgtgtgcaagcactctatggtggataggggaggaggaaacggatgtggcctgttcg
gcaagggcggcatcgtgacctgcgccatgtttcggtgtaagaagaacatggagggcaaggtggtg
cagcctgagaatctggagtacaccatcgtgatcacaccacacagcggagaggagcacgcagtggg
aaatgacaccggcaagcacggcaaggagatcaagatcacaccacagagctccatcaccgaggccg
agctgacaggctatggcacagtgaccatggagtgctccccaaggaccggcctggatttcaacgag
atggtgctgctgcagatggagaataaggcctggctggtgcaccgccagtggtttctggacctgcc
actgccatggctgcctggagcagatacccagggctccaactggatccagaaggagacactggtga
ccttcaagaatccacacgccaagaagcaggacgtggtggtgctgggctctcaggagggagcaatg
cacacagccctgaccggagccacagagatccagatgtctagcggcaatctgctgttcaccggcca
cctgaagtgccggctgagaatggataagctgcagctgaagggcatgagctactccatgtgcaccg
gcaagtttaaggtggtgaaggagatcgccgagacacagcacggcacaatcgtgatcagggtgcag
tatgagggcgacggcagcccttgtaagatcccattcgagatcatggatctggagaagcggcacgt
gctgggcagactgatcaccgtgaacccaafcgtgacagagaaggactctcccgtgaatatcgagg
ccgagcccccttttggcgatagctacatcatcatcggcgtggagcccggccagctgaagctgaac
tggttcaagaagtaa
DNA sequence of mutant E protein N8R plus 5′UTR + 3′ UTR
(SEQ ID NO: 11)
Gggaaataagagagaaaagaagagtaagaagaaatataagagccaccatggagacagacacactc
ctgctatgggtactgctgctctgggttccaggttccaccggtgacatgaggtgtattggaatgtc
tagacgggattttgtggagggagtctctggcgggagttgggtggatattgtcctggagcacggaa
gctgcgtgaccacaatggccaagaacaagcccaccctggacttcgagctgatcaagaccgaggcc
aagcagcctgccacactgcggaagtactgcatcgaggccaagctgaccaacaccacaaccgagag
cagatgtcccacacagggcgagccttccctgaatgaggagcaggacaagcggttcgtgtgcaagc
actctatggtggataggggatggggaaacggatgtggcctgttcggcaagggcggcatcgtgacc
tgcgccatgtttcggtgtaagaagaacatggagggcaaggggtgcagcctgagaatctggagtac
accatcgtgatcacaccacacagcggagaggagcacgcagtgggaaatgacaccggcaagcacgg
caaggagatcaagatcacaccacagagctccatcaccgaggccgagctgacaggctatggcacag
tgaccatggagtgctccccaaggaccggcctggatttcaacgagatggtgctgctgcagatggag
aataaggcctggctggtgcaccgccagtggtttctggacctgccactgccatggctgcctggagc
agatacccagggctccaactggatccagaaggagacactggtgaccttcaagaatccacacgcca
agaagcaggacgtggtggtgctgggctctcaggagggagcaatgcacacagccctgaccggagcc
acagagatccagatgtctagcggcaatctgctgttcaccggccacctgaagtgccggctgagaat
ggataagctgcagctgaagggcatgagctactccatgtgcaccggcaagtttaaggtggtgaagg
agatcgccgagacacagcacggcacaatcgtgatcagggtgcagtatgagggcgacggcagccct
tgtaagatcccattcgagatcatggatctggagaagcggcacgtgctgggcagactgatcaccgt
gaacccaatcgtgacagagaaggactctcccgtgaatatcgaggccgagcccccttttggcgata
gctacatcatcatcggcgtggagcccggccagctgaagctgaactggttcaagaagtaaggtacc
tgataataggctggagcctcggtggccatgcttcttgccccttgggcctccccccagcccctcct
ccccttcctgcacccgtacccccgtggtctttgaataaagtctgagtgggcggcaaaaaaaaaaa
aaaaaaaaaaaaaaaaaaaaaaaaaaaaaaaaaaaaaaaaaaaaaaaaaaaaaaaaaaaaaaaaa
aaaaaaaaaaaaaaaaaaaaaaaa
DNA sequence of mutant E protein W101G plus 5′UTR +3′ UTR
(SEQ ID NO: 12)
Gggaaataagagagaaaagaagagtaagaagaaatataagagccaccatggagacagacacactc
ctgctatgggtactgctgctctgggttccaggttccaccggtgacatgaggtgtattggaatgtc
taaccgggattttgtggagggagtctctgggggagttgggtggatattgtcctggagcacggaag
ctgcgtgaccacaatggccaagaacaagcccaccctggacttcgagctgatcaagaccgaggcca
agcagcctgccacactgcggaagtactgcatcgaggccaagctgaccaacaccacaaccgagagc
agatgtcccacacagggcgagccttccctgaatgaggagcaggacaagcggttcgtgtgcaagca
ctctatggtggataggggaggaggaaacggatgtggcctgttcggcaagggcggcatcgtgacct
gcgccatgtttcggtgtaagaagaacatggagggcaaggtggtgcagcctgagaatctggagtac
accatcgtgatcacaccacacagcggagaggagcacgcagtgggaaatgacaccggcaagcacgg
caaggagatcaagatcacaccacagagctccatcaccgaggccgagctgacaggctatggcacag
tgaccatggagtgctccccaaggaccggcctggatttcaacgagatggtgctgctgcagatggag
aataaggcctggctggtgcaccgccagtggtttctggacctgccactgccatggctgcctggagc
agatacccagggctccaactggatccagaaggagacactggtgaccttcaagaatccacacgcca
agaagcaggacgtggtggtgctgggctctcaggagggagcaatgcacacagccctgaccggagcc
acagagatccagatgtctagcggcaatctgctgttcaccggccacctgaagtgccggctgagaat
ggataagctgcagctgaagggcatgagctactccatgtgcaccggcaagtttaaggtggtgaagg
agatcgccgagacacagcacggcacaatcgtgatcagggtgcagtatgagggcgacggcagccct
tgtaagatcccattcgagatcatggatctggagaagcggcacgtgctgggcagactgatcaccgt
gaacccaatcgtgacagagaaggactctcccgtgaatatcgaggccgagcccccttttggcgata
gctacatcatcatcggcgtggagcccggccagctgaagctgaactggttcaagaagtaaggtacc
tgataataggctggagcctcggtggccatgcttcttgccccttgggcctccccccagcccctcct
ccccttcctgcacccgtacccccgtggtctttgaataaagtctgagtgggcggcaaaaaaaaaaa
aaaaaaaaaaaaaaaaaaaaaaaaaaaaaaaaaaaaaaaaaaaaaaaaaaaaaaaaaaaaaaaaa
aaaaaaaaaaaaaaaaaaaaaaaa
DNA sequence of mutant E protein N8R + W101G plus 5′UTR + 3′ UTR
(SEQ ID NO: 13)
gggaaataagagagaaaagaagagtaagaagaaatataagagccaccatggagacagacacactc
ctgctatgggtactgctgctctgggttccaggttccaccggtgacatgaggtgtattggaatgtc
tagacgggattttgtggagggagtctctggcgggagttgggtggatattgtcctggagcacggaa
gctgcgtgaccacaatggccaagaacaagcccaccctggacttcgagctgatcaagaccgaggcc
aagcagcctgccacactgcggaagtactgcatcgaggccaagctgaccaacaccacaaccgagag
cagatgtcccacacagggcgagccttccctgaatgaggagcaggacaagcggttcgtgtgcaagc
actctatggtggataggggaggaggaaacggatgtggcctgttcggcaagggcggcatcgtgacc
tgcgccatgtttcggtgtaagaagaacatggagggcaaggtggtgcagcctgagaatctggagta
caccatcgtgatcacaccacacagcggagaggagcacgcagtgggaaatgacaccggcaagcacg
gcaaggagatcaagatcacaccacagagctccatcaccgaggccgagctgacaggctatggcaca
gtgaccatggagtgctccccaaggaccggcctggatttcaacgagatggtgctgctgcagatgga
gaataaggcctggctggtgcaccgccagtggtttctggacctgccactgccatggctgcctggag
cagatacccagggctccaactggatccagaaggagacactggtgaccttcaagaatccacacgcc
aagaagcaggacgtggtggtgctgggctctcaggagggagcaatgcacacagccctgaccggagc
cacagagatccagatgtctagcggcaatctgctgttcaccggccacctgaagtgccggctgagaa
tggataagctgcagctgaagggcatgagctactccatgtgcaccggcaagtttaaggtggtgaag
gagatcgccgagacacagcacggcacaatcgtgatcagggtgcagtatgagggcgacggcagccc
ttgtaagatcccattcgagatcatggatctggagaagcggcacgtgctgggcagactgatcaccg
tgaacccaatcgtgacagagaaggactctcccgtgaatatcgaggccgagcccccttttggcgat
agctacatcatcatcggcgtggagcccggccagctgaagctgaactggttcaagaagtaaggtac
ctgataataggctggagcctcggtggccatgcttcttgccccttgggcctccccccagcccctcc
tccccttcctgcacccgtacccccgtggtctttgaataaagtctgagtgggcggcaaaaaaaaaa
aaaaaaaaaaaaaaaaaaaaaaaaaaaaaaaaaaaaaaaaaaaaaaaaaaaaaaaaaaaaaaaaa
aaaaaaaaaaaaaaaaaaaaaaaaa

### REFERENCES

1. Amorim, J. H., and Birbrair, A. (2022). Dengue vaccines: where are we now and where we are going? The Lancet Infectious Diseases 22, 756-757.
2. August, A., Attarwala, H. Z., Himansu, S., Kalidindi, S., Lu, S., Pajon, R., Han, S., Lecerf, J.-M., Tomassini, J. E., Hard, M., et al. (2021). A phase 1 trial of lipid-encapsulated mRNA encoding a monoclonal antibody with neutralizing activity against Chikungunya virus. Nature Medicine 27, 2224-2233.
3. Berneck, B. S., Rockstroh, A., Fertey, J., Grunwald, T., and Ulbert, S. (2020). A Recombinant Zika Virus Envelope Protein with Mutations in the Conserved Fusion Loop Leads to Reduced Antibody Cross-Reactivity upon Vaccination. Vaccines 8.
4. Bhatt, P., Sabeena, S. P., Varma, M., and Arunkumar, G. (2021). Current Understanding of the Pathogenesis of Dengue Virus Infection. Current microbiology 78, 17-32.
5. Chaudhary, N., Weissman, D., and Whitehead, K. A. (2021). mRNA vaccines for infectious diseases: principles, delivery and clinical translation. Nature Reviews Drug Discovery 20, 817-838.
6. Costin, J. M., Zaitseva, E., Kahle, K. M., Nicholson, C. O., Rowe, D. K., Graham, A. S., Bazzone, L. E., Hogancamp, G., Sierra, M. F., Fong, R. H., et al. (2013). Mechanistic Study of Broadly Neutralizing Human Monoclonal Antibodies against Dengue Virus That Target the Fusion Loop. Journal of virology 87, 52-66
7. Ge, N., Sun, J., Liu, Z., Shu, J., Yan, H., Kou, Z., Wei, Y., and Jin, X. (2022). An mRNA vaccine encoding Chikungunya virus E2-E1 protein elicits robust neutralizing antibody responses and CTL immune responses. Virologica Sinica 37, 266-276.
8. Guzman, M. G., Gubler, D. J., Izquierdo, A., Martinez, E., and Halstead, S. B. (2016). Dengue infection. Nature Reviews Disease Primers 2, 16055.
9, Guzman, M. G., Halstead, S. B., Artsob, H., Buchy, P. Farrar, J., Gubler, D. J., Hunsperger, E., Kroeger, A., Margolis, H. S., Martinez, E., et al. (2010). Dengue: a continuing global threat. Nature Reviews Microbiology 8, S7-S16.
10. He, L., Sun, W., Yang, L., Liu, W., and Li, J. (2022). A multiple-target mRNA-LNP vaccine induces protective immunity against experimental multi-serotype DENV in mice. Virologica Sinica 37, 746-757.
11. Henein, S., Adams, C., Bonaparte, M., Moser, J. M., Munteanu, A., Baric, R., and de Silva, A. M. (2021). Dengue vaccine breakthrough infections reveal properties of neutralizing antibodies linked to protection. The Journal of clinical investigation 131.
12. Hsu, F. F., Liang, K. H., Kumari, M., Chen, W. Y., Lin, H. T., Cheng, C. M., Tao, M. H., and Wu, H. C. (2022). An efficient approach for SARS-COV-2 monoclonal antibody production via modified mRNA-LNP immunization. Int J Pharm 627, 122256.
13. Khetarpal, N., and Khanna, I. (2016). Dengue Fever: Causes, Complications, and Vaccine Strategies. Journal of immunology research 2016, 6803098.
14. Kudlacek, S. T., Metz, S., Thiono, D., Payne, A. M., Phan, T. T. N., Tian, S., Forsberg, L. J., Maguire, J., Seim, I., Zhang, S., et al. (2021). Designed, highly expressing, thermostable dengue virus 2 envelope protein dimers elicit quaternary epitope antibodies. Science Advances 7, eabg4084.
15. Lai, C. Y., Tsai, W. Y., Lin, S. R., Kao, C. L., Hu, H. P., King, C. C., Wu, H. C., Chang, G. J., and Wang, W. K. (2008). Antibodies to envelope glycoprotein of dengue virus during the natural course of infection are predominantly cross-reactive and recognize epitopes containing highly conserved residues at the fusion loop of domain II. Journal of virology 82, 6631-6643.
16. Mallapaty, S. (2022). Dengue vaccine poised for roll-out but safety concerns linger. Nature.
17. Messer, W. B., de Alwis, R., Yount, B. L., Royal, S. R., Huynh, J. P., Smith, S. A., Crowe, J. E., Doranz, B. J., Kahle, K. M., Pfaff, J. M., et al. (2014). Dengue virus envelope protein domain I/II hinge determines long-lived serotype-specific dengue immunity. Proceedings of the National Academy of Sciences 111, 1939-1944.
18. Pardi, N., Hogan, M. J., Porter, F. W., and Weissman, D. (2018), mRNA vaccines—a new era in vaccinology. Nature reviews Drug discovery 17, 261-279.
19. Pinheiro-Michelsen, J. R., Souza, R., Santana, I. V. R., da Silva, P. S., Mendez, E. C., Luiz, W. B., and Amorim, J. H. (2020). Anti-dengue Vaccines: From Development to Clinical Trials. Frontiers in immunology 17, 1252.
20. Rajamanonmani, R., Nkenfou, C., Clancy, P., Yau, Y. H., Shochat, S. G., Sukupolvi-Petty, S., Schul, W., Diamond, M. S., Vasudevan, S. G., and Lescar, J. (2009). On a mouse monoclonal antibody that neutralizes all four dengue virus serotypes. The Journal of general virology 90, 799-809.
21. Rockstroh, A., Barzon, L., Pacenti, M., Palù, G., Niedrig, M., and Ulbert, S. (2015). Recombinant Envelope-Proteins with Mutations in the Conserved Fusion Loop Allow Specific Serological Diagnosis of Dengue-Infections. PLOS Negl Trop Dis 9, e0004218.
22. Shen, W.-F., Galula, J. U., Liu, J.-H., Liao, M.-Y., Huang, C.-H., Wang, Y.-C., Wu, H.-C., Liang, J.-J., Lin, Y.-L., Whitney, M. T., et al. (2018). Epitope resurfacing on dengue virus-like particle vaccine preparation to induce broad neutralizing antibody. eLife 7, e38970.
23. Tang, C.-T., Li. P.-C., Liu, I. J., Liao, M.-Y., Chiu, C.-Y., Chao, D.-Y., and Wu, H.-C. (2015). An Epitope-Substituted DNA Vaccine Improves Safety and Immunogenicity against Dengue Virus Type 2. PLOS Neglected Tropical Diseases 9, e0003903.
24. Thomas, S. J., and Yoon, I. K. (2019). A review of Dengvaxia®: development to deployment. Human vaccines & immunotherapeutics 15, 2295-2314.
25. Torres-Flores, J. M., Reyes-Sandoval, A., and Salazar, M. I. (2022). Dengue Vaccines: An Update. BioDrugs 36, 325-336.
26. Tully, D., and Griffiths, C. L. (2021). Dengvaxia: the world's first vaccine for prevention of secondary dengue. Therapeutic advances in vaccines and immunotherapy 9, 25151355211015839.
27. Valentin, A., Bergamaschi, C., Rosati, M., Angel, M., Burns, R., Agarwal, M., Gergen, J., Petsch, B., Oostvogels, L., Loeliger, E., et al. (2022). Comparative immunogenicity of an mRNA/LNP and a DNA vaccine targeting HIV gag conserved elements in macaques. Frontiers in immunology 13.
28. VanBlargan, L. A., Himansu, S., Foreman, B. M., Ebel, G. D., Pierson, T. C., and Diamond, M. S. (2018). An mRNA Vaccine Protects Mice against Multiple Tick-Transmitted Flavivirus Infections. Cell reports 25, 3382-3392.e3383.
29. WHO (2022). Dengue and severe dengue. In.
30. Wilder-Smith, A. (2020). Dengue vaccine development: status and future. Bundesgesundheitsblatt, Gesundheitsforschung, Gesundheitsschutz 63, 40-44.
31. Wollner, C. J., and Richner, J. M. (2021). mRNA Vaccines against Flaviviruses, Vaccines 9.
32. Wollner, C. J., Richner, M., Hassert, M. A., Pinto, A. K., Brien, J. D., and Richner, J. M. (2021). A Dengue Virus Serotype 1 mRNA-LNP Vaccine Elicits Protective Immune Responses. Journal of virology 95.
33. Zhang, M., Sun, J., Li, M., and Jin, X. (2020). Modified mRNA-LNP Vaccines Confer Protection against Experimental DENV-2 Infection in Mice. Molecular therapy Methods & clinical development 18, 702-712.

1. A composition, which comprises a messenger ribonucleic acid (mRNA) comprising an open reading frame encoding a dengue virus envelop (E) protein variant formulated in a lipid nanoparticle, wherein the E protein variant comprises amino acid mutation at position 8 and/or position 101 of SEQ ID NO: 2.

2. The composition of claim 1, wherein the one or more amino acid mutation is selected from the group consisting of
(a) an amino acid substitution of the 8^{th} Asn residue;
(b) an amino acid substitution of the 101^{th} Try residue; and
(c) an amino acid substitution of the 8^{th} Asn residue and an amino acid substitution of the 101^{th} Try residue.

3. The composition of claim 1, wherein the one or more amino acid mutation is selected from the group consisting of
(a) an amino acid substitution of the 8^{th} Asn residue with Arg (R);
(b) an amino acid substitution of the 101^{th} Try residue with Gly (G); and
(c) an amino acid substitution of the 8^{th} Asn (N) residue with Arg (R) and an amino acid substitution of the 101^{th} Try (W) residue with Gly.

4. The composition of claim 1, wherein
the mRNA encodes SEQ ID NO: 5;
the mRNA encodes SEQ ID NO: 6; or
the mRNA encodes SEQ ID NO: 7.

5. The composition of claim 1, wherein
the mRNA comprises a transcript from SEQ ID NO: 8;
the mRNA comprises a transcript from SEQ ID NO: 9; or
the mRNA comprises a transcript from SEQ ID NO: 10.

6. The composition of claim 1, wherein the mRNA
further comprises
a 5′ untranslated region (UTR) and a 3′ UTR.

7. The composition of claim 1, wherein
the mRNA comprises a transcript from SEQ ID NO: 11;
the mRNA comprises a transcript from SEQ ID NO: 12; or
the mRNA comprises a transcript from SEQ ID NO: 13.

8. The composition of claim 1, wherein the mRNA further comprises
a 5′ cap analog; and/or
a poly(A) tail.

9. The composition of claim 8, wherein the 5′ cap analog is 7 mG(5′)ppp(5′)NlmpNp.

10. The composition of claim 1, wherein the mRNA comprises a chemical modification.

11. The composition of claim 10, wherein the chemical modification is a 1-methylpseudouridine modification.

12. The composition of claim 1, wherein the lipid nanoparticle comprises a cationic lipid, a non-cationic lipid, a sterol and a polyethylene glycol (PEG)-modified lipid.

13. The composition of claim 1, wherein the lipid nanoparticle comprises 20-70% cationic lipid, 5-45% non-cationic lipid, 20-55% sterol, and 0.5-15% PEG-modified lipid.

14. The composition of claim 1, wherein the lipid nanoparticle comprises 50% cationic lipid, 10% non-cationic lipid, 38.5% sterol and 1.5% PEG-modified lipid.

15. The composition of claim 1, wherein the lipid nanoparticle has a diameter from about 10 to about 500 nm.

16. The composition of claim 1, wherein the lipid nanoparticle comprises a lipid bilayer surrounding a core where the mRNA is encapsulated.

17. A vaccine against dengue virus infection which comprises an effective amount of a composition of claim 1 and a physiologically acceptable vehicle.

18. The vaccine of claim 17, wherein the dengue virus infection is caused by dengue virus serotype 1 (DENV1), dengue virus serotype 2 (DENV2), dengue virus serotype 3 (DENV3) and/or dengue virus serotype 4 (DENV4).

19. The vaccine of claim 17, wherein the amount of the composition is effective in inducing neutralizing antibodies against the dengue virus infection, and/or alleviating antibody dependent enhancement of the dengue virus infection.

20. A composition of claim 1, suitable for use in immunization against dengue virus infection in a subject in need thereof.

21. The composition of claim 20, wherein the dengue virus infection is caused by dengue virus serotype 1 (DENV1), dengue virus serotype 2 (DENV2), dengue virus serotype 3 (DENV3) and/or dengue virus serotype 4 (DENV4).

22. The composition of claim 20, wherein the immunization incudes neutralizing antibodies against the dengue virus infection and/or alleviates antibody dependent enhancement of the dengue virus infection.

23. (canceled)

24. (canceled)

25. (canceled)

26. A method for immunization against dengue virus infection in a subject in need thereof, comprising:
administering to the subject in need thereof a therapeutically effective amount of a composition of claim 1.

27. The method of claim 26, wherein the amount of the composition or vaccine is effective in inducing neutralizing antibodies against the dengue virus infection and/or alleviating antibody dependent enhancement of the dengue virus infection in the subject.

28. The method of claim 26, wherein the composition is administered to the subject once or more than once.
